(19) United States
(12) Patent Application Publication
(10) Pub. No.: US 2003/0099969 A1
(43) Pub. Date: May 29, 2003

(54) Ipaf, an ice-protease activating factor

(76) Inventor: Emad S. Alnemri, Ambler, PA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/156,733

(22) Filed: May 24, 2002

Related U.S. Application Data
(60) Provisional application No. 60/293,597, filed on May 25, 2001.

(51) Int. Cl. C12Q 1/68; C07H 21/04; C12N 9/99; C12P 21/02; C12N 5/06

(52) U.S. Cl. 435/6; 435/69.2; 435/184; 435/320.1; 435/325; 536/23.2

(57) ABSTRACT

Ipaf nucleic acid molecules and encoded polypeptides are provided. Also provided are methods of using Ipaf polypeptides and nucleic acid molecules. The Invention also provides methods of screening for inhibitors or enhancers of Ipaf-mediated apoptosis or procaspase-1 processing. The invention further provides production, isolation and purification of Ipaf polypeptides as well as antibodies against said polypeptides. In addition, the invention provides compositions of Ipaf polypeptides, anti-Ipaf antibodies, and/or Ipaf nucleic acid molecules that can be utilized for diagnostic or therapeutic purposes.

### STATEMENT REGARDING FEDERALLY SPONSORED RESEARCH OR DEVELOPMENT

The present invention was made in part with funds provided by the United States Government under National Institutes of Health Research grant AG 14357. Accordingly, the United States Government may have certain rights to this invention.

### BACKGROUND OF THE INVENTION

1. Field of the Invention

The present invention relates generally to polynucleic acids and products encoded thereby which regulate apoptosis, proliferation, and inflammation. More specifically, the present invention discloses nucleic acids and polypeptides corresponding to Ipaf, a novel apoptotic protein, and methods of using the same to identify apoptotic modulators as well as to diagnose and treat diseases and disorders involving cell proliferation, apoptosis, inflammation or immune response.

2. Description of the Related Art

Apoptosis is a physiological cellular process of programmed cell death that is essential for normal development and homeostasis of multicellular organisms. Apoptosis is involved in a range of physiological processes such as embryonic development, immune cell regulation and normal cellular turnover. Thus, dysfunction or loss of regulated apoptosis can lead to a variety of pathological disease states. For example, the loss of apoptosis can lead to the pathological accumulation of self-reactive lymphocytes that occurs with many autoimmune diseases. Inappropriate loss or inhibition of apoptosis can also lead to the accumulation of virally infected cells and hyperproliferative cells such as neoplastic or tumor cells. Similarly, the inappropriate activation of apoptosis can also contribute to a variety of pathological disease states including, for example, acquired immunodeficiency syndrome (AIDS), neurodegenerative diseases and ischemic injury. Treatments that are specifically designed to modulate the apoptotic pathways in these and other pathological conditions can alter the natural progression of many of these diseases.

Apoptosis is mediated by diverse signals and complex interactions of cellular gene products. Several gene families and products that modulate the apoptotic process have now been identified. One family is the aspartate-specific cysteine proteases (“caspases”). The human caspase family includes, for example, Ced-3, human ICE (interleukin-1-β converting enzyme) (caspase-1), ICH-1 (caspase-2), CPP32 (caspase-3), ICEreIII (caspase-4), ICEreIIII (caspase-5), Mch2 (caspase-6), ICE-LAP3 (caspase-7), Mch5 (caspase-8) ICE-LAP6 (caspase-9), Mch4 (caspase-10), caspases 11-14, and others.

Caspases are cysteine proteases (named for a cysteine residue in the active site) that cleaves substrates at Asp-X bonds. Caspases are primarily produced as inactive zymogens that require proteolytic cleavage at specific internal aspartate residues for activation. The primary gene product is arranged such that the N-terminal peptide (prodomain) precedes a large subunit domain, which precedes a small subunit domain. The large subunit contains the conserved active site pentapeptide QACXG (X=R, Q, G) (SEQ ID NO: 11) which contains the nucleophilic cysteine residue. The small subunit contains residues that bind the Asp carboxylate side chain and others that determine-substrate specificity. Cleavage of a caspase yields the two subunits, the large (generally approximately 20 kD) and the small (generally approximately 10 kD) subunit that associate non-covalently to form a heterodimer, and, in some caspases, an N-terminal peptide of varying length. The heterodimer may combine non-covalently to form a tetramer.

Caspase zymogens are themselves substrates for caspases. Inspection of the interdomain linkages in each zymogen reveals target sites (i.e. protease sites) that indicate a hierarchical relationship of caspase activation. By analyzing such pathways, it has been demonstrated that caspases are required for apoptosis to occur. Moreover, caspases appear to be necessary for the accurate and limited proteolytic events which are the hallmark of classic apoptosis (see Salvesen and Dixit, Cell 91:443-446, 1997). Once activated, most caspases can process and activate their own and other inactive procaspases in vitro (Fernandes-Alnemri et al., Proc. Natl. Acad. Sci. USA 93:7464-7469, 1996; Srinivasula et al., Proc. Natl. Acad. Sci. USA 93:13706-13711, 1996). This characteristic suggests that caspases implicated in apoptosis may execute the apoptotic program through a cascade of sequential activation of initiators and executioner procaspases (Salvesen and Dixit, Cell 91:443-446, 1997). The initiators are responsible for processing and activation of the executioners. The executioners are responsible for proteolytic cleavage of a number of cellular proteins leading to the characteristic morphological changes and DNA fragmentation that are often associated with apoptosis (reviewed by Cohen, Biochem. J. 326:1-16, 1997; Henkart, Immunity 4:195-201, 1996; Martin and Green, Cell 82:349-352, 1995; Nicholson and Thornberry, TIBS 257:299-306, 1997; Porter et al., BioEssays 19:501-507, 1997; Salvesen and Dixit, Cell91:443-446, 1997).

Caspase-1, also known as interleukin-1β converting enzyme, is known for its function of proteolytic processing of the precursors of proinflammatory cytokines such as interleukin-1β into active cytokines (Cryns and Yuan, Genes Dev. 12: 1551-1570, 1998). Thus, caspase-1 plays a key role in inflammation. Further studies have demonstrated that caspase-1 possesses an important role in programmed cell death as caspase-1 knockout mice demonstrate a partial defect in apoptosis (Li et al., Cell 80:401-411, 1995). More recent studies show that caspase-1 is necessary for apoptosis induced by external signals such as Fas/FasL. More importantly, caspase-1 acts as an upstream activator of the cascade of caspase activations during apoptosis (Aiba-Masago et al., Cell Signal. 13:617-24, 2001). Caspase-1 is synthesized as a single chain polypeptide zymogen (procaspase-1). Procaspase-1 is expressed in many tissues as an inactive proenzyme polypeptide of 404 amino acids and a relative molecular mass of 45 kD (Alnemri et al., J. Biol. Chem. 270:4312-4317, 1995). Caspase-1 is produced after proteolytic cleavage of the procaspase-1 to generate two subunits of molecular mass 20 and 10 kD, known as p20 and p10 subunits (Alnemri et al., J. Biol. Chem. 270:4312-4317, 1995). Prior studies show that RICK (RIP-like interacting CLARP kinase) is implicated in the regulation of caspase-1 activity (Humke et al., Cell 103:99-111, 2000). However, the exact mechanism and molecules that regulate the activation of caspase-1 have yet to be fully understood.

Therefore, there exists a need in the art to identify genes and gene products related to the activity of caspase-1 as well as its role in inflammation and apoptosis. To this end there exists a clear need to identify inhibitors and enhancers of the functionality of said gene products. The present invention satisfies these needs and provides other related advantages.

### BRIEF SUMMARY OF THE INVENTION

The present invention provides novel Ipaf polypeptides or functional fragments thereof and isolated nucleic acid molecules encoding such polypeptides. The present invention also provides Ipaf-encoding nucleic acid molecules in vectors, host cells, gene delivery vehicles, and kits, as well as antibodies directed to naturally and recombinantly expressed Ipaf polypeptides or immunoreactive fragments. The present invention further provides antisense of the Ipaf polynucleotides and therapeutic compositions. The present invention additionally provides methods of inducing apoptosis, of treating and diagnosing certain diseases, and for identifying an agent that alters the activity of the Ipaf polypeptide.

In one aspect, the present invention provides an isolated nucleic acid molecule that comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or Ipaf of SEQ ID NO: 2. In one embodiment, the encoded Ipaf is a human Ipaf. In a further embodiment, the human Ipaf is encoded by the nucleotide sequence of SEQ ID NO: 1, SEQ ID NO:14, or a degenerate variant of either. In another embodiment, the encoded Ipaf polypeptide or functional fragment thereof comprises an amino acid sequence of SEQ ID NO: 2.

Another embodiment of the present invention provides an isolated nucleic acid molecule that comprises a polynucleotide sequence encoding an Ipaf polypeptide or functional fragment thereof, wherein the polynucleotide sequence encodes a CARD domain of an Ipaf polypeptide, said CARD domain having at least 90% identity with amino acids 1-88 of SEQ ID NO: 2, wherein said CARD domain mediates dimerization between the Ipaf proteins or with caspase-1/procaspase-1. In one further embodiment, the polynucleotide sequence encoding the CARD domain comprises nucleotides 1-264 of SEQ ID NO: 1 or a degenerate variant thereof. In another further embodiment, the encoded CARD domain comprises amino acids 1-88 of SEQ ID NO: 2.

In another embodiment, the present invention provides an isolated nucleic acid molecule that comprises a polynucleotide sequence encoding an Ipaf polypeptide or functional fragment thereof, wherein the polynucleotide sequence encodes a NBD domain of an Ipaf polypeptide, said NBD domain having at least 90% identity with amino acids 163-457 of SEQ ID NO: 2, wherein said NBD domain is capable of binding nucleotides. In one further embodiment, the polynucleotide sequence encoding the NBD domain comprises nucleotides 487-1371 of SEQ ID NO: 1 or a degenerate variant thereof. In another further embodiment, the encoded NBD domain comprises amino acids 163-457 of SEQ ID NO: 2.

It is another embodiment of the invention to provide an isolated nucleic acid molecule that comprises a polynucleotide sequence encoding an Ipaf polypeptide or functional fragment thereof, wherein the polynucleotide sequence encodes an LRR domain of an Ipaf polypeptide, said LRR domain having at least 90% identity with amino acids 656-1024 of SEQ ID NO: 2, wherein said LRR domain is capable of interacting specifically with another LRR domain, thereby mediating protein-protein interactions. In one further embodiment, the polynucleotide sequence encoding the LRR domain comprises nucleotides 1966-3072 of SEQ ID NO: 1 or a degenerate variant thereof. In another further embodiment, the encoded LRR domain comprises amino acids 656-1024 of SEQ ID NO: 2.

It is another embodiment of the invention to provide an isolated nucleic acid molecule that comprises a polynucleotide sequence encoding an Ipaf polypeptide or functional fragment thereof, wherein the polynucleotide sequence encodes a functional fragment of an Ipaf polypeptide, wherein the Ipaf functional fragment comprises amino acids 1-256 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the polynucleotide sequence encoding the functional fragment comprises nucleotides 1-768 of SEQ ID NO: 1 or a degenerate variant thereof.

It is another embodiment of the invention to provide an isolated nucleic acid molecule that comprises a polynucleotide sequence encoding an Ipaf polypeptide or functional fragment thereof, wherein the polynucleotide sequence encodes a functional fragment of an Ipaf polypeptide, wherein the Ipaf functional fragment comprises amino acids 1-328 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the polynucleotide sequence encoding the functional fragment comprises nucleotides 1-984 of SEQ ID NO: 1 or a degenerate variant thereof.

It is another embodiment of the invention to provide an isolated nucleic acid molecule that comprises a polynucleotide sequence encoding an Ipaf polypeptide or functional fragment thereof, wherein the polynucleotide sequence encodes a functional fragment of an Ipaf polypeptide, wherein the Ipaf functional fragment comprises amino acids 1-557 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the polynucleotide sequence encoding the functional fragment comprises nucleotides 1-1671 of SEQ ID NO: 1 or a degenerate variant thereof.

It is another embodiment of the invention to provide an isolated nucleic acid molecule that comprises a polynucleotide sequence encoding an Ipaf polypeptide or functional fragment thereof, wherein the polynucleotide sequence encodes a functional fragment of an Ipaf polypeptide, wherein the Ipaf functional fragment comprises amino acids 1-601 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the polynucleotide sequence encoding the functional fragment comprises nucleotides 1-1803 of SEQ ID NO: 1 or a degenerate variant thereof.

It is another embodiment of the invention to provide an isolated nucleic acid molecule that comprises a polynucleotide sequence encoding an Ipaf polypeptide or functional fragment thereof, wherein the polynucleotide sequence encodes a functional fragment of an Ipaf polypeptide, wherein the Ipaf functional fragment comprises amino acids 1-661 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the polynucleotide sequence encoding the functional fragment comprises nucleotides 1-1983 of SEQ ID NO: 1 or a degenerate variant thereof.

In another aspect, the present invention provides an isolated nucleic acid molecule encoding an Ipaf polypeptide or functional fragment thereof, comprising a polynucleotide sequence having at least 70% identity with SEQ ID NO: 1, wherein said Ipaf polypeptide or functional fragment thereof induces or suppresses procaspase-1 activation or oligomerizes with procaspase-1 or Ipaf of SEQ ID NO: 2. In one embodiment, the polynucleotide sequence comprises a nucleotide sequence of SEQ ID NO: 1 or a degenerate variant thereof. In another embodiment, the polynucleotide sequence comprises at least 20 contiguous nucleotide residues of the sequence provided in SEQ ID NO: 1. In yet another embodiment, the polynucleotide sequence comprises at least 50 contiguous nucleotide residues of the sequence provided in SEQ ID NO: 1. In still another embodiment, the polynucleotide sequence comprises at least 100 contiguous nucleotide residues of the sequence provided in SEQ ID NO: 1.

In another aspect, the present invention provides an expression vector that comprises a promoter, and an isolated nucleic acid molecule, wherein the nucleic acid molecule is operably linked to the promoter, and wherein the nucleic acid molecule comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1. In one embodiment, the promoter is a constitutive or inducible promoter.

In another aspect, the present invention provides an isolated nucleic acid molecule that comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1; wherein the polynucleotide sequence encoding the Ipaf or a functional fragment thereof is fused to a heterologous nucleotide sequence. In one embodiment, the heterologous nucleotide sequence is selected from the group consisting of β-galactosidase, β-glucuronidase, green fluorescent protein (GFP), blue fluorescent protein (BFP), glutathione-S-transferase (GST), luciferase, horseradish perozidase (HRP), and chloramphenicol acetyltransferase (CAT).

In another aspect, the present invention provides an isolated nucleic acid molecule that comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1; wherein the polynucleotide sequence encoding the Ipaf or a fragment thereof is fused in frame at either the N-terminal or C-terminal of the Ipaf with a nucleotide sequence encoding a tag, wherein the tag is selected from the group consisting of histidine (His) tags, Asp-Tyr-Lys-Asp-Asp-Asp-Asp-Lys (FLAG) tag (SEQ ID NO: 12), Met-Ala-Ser-Met-Thr-Gly-Gly-Gln-Gln-Met-Gly (T7) tag (SEQ ID NO: 13), influenza hemagglutinin (HA) tags, Myc tags, VSV-G tags, and thioredoxin (Trx) tags.

In another aspect, the present invention provides a host cell that contains an expression vector that comprises a promoter, and an isolated nucleic acid molecule, wherein the nucleic acid molecule is operably linked to the promoter, and wherein the nucleic acid molecule comprises a polynucleotide sequence encoding an Ipaf polypeptide or functional fragment thereof, wherein said Ipaf polypeptide or functional fragment thereof has at least 90% identity with SEQ ID NO 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1. In certain embodiments, the host cell is selected from the group consisting of a bacterium, a yeast cell, a nematode cell, an insect cell, a plant cell and a mammalian cell.

In another aspect, the present invention provides an isolated Ipaf polypeptide or functional fragment thereof that has at least 90% amino acid identity with SEQ ID NO: 2, wherein said Ipaf polypeptide or functional fragment thereof induces or suppresses procaspase-1 activation or oligomerizes with procaspase-1 or Ipaf of SEQ ID NO: 2. In one embodiment, the Ipaf is a human Ipaf. In a further embodiment, the Ipaf polypeptide comprises the amino acid sequence presented in SEQ ID NO: 2. In another embodiment, the Ipaf polypeptide is encoded by SEQ ID NO: 1 or a degenerate variant thereof or fragment thereof.

In another embodiment, the present invention provides an isolated Ipaf polypeptide or functional fragment thereof, wherein the Ipaf polypeptide or functional fragment thereof comprises a CARD domain, said CARD domain having at least 90% identity with amino acids 1-88 of SEQ ID NO: 2; and wherein said CARD domain mediates dimerization between the Ipaf polypeptide or functional fragment thereof derived from SEQ ID NO: 2 or with caspase-1/procaspase-1. In a further embodiment, the CARD domain comprises amino acids 1-88 of SEQ ID NO: 2. In another further embodiment, the CARD domain is encoded by nucleotides 1-264 of SEQ ID NO: 1 or a degenerate variant thereof.

In another embodiment, the present invention provides an isolated Ipaf polypeptide or functional fragment thereof, wherein the Ipaf polypeptide or functional fragment thereof comprises a NBD domain, said NBD domain having at least 90% identity with amino acids 163-457 of SEQ ID NO: 2, wherein said NBD domain is capable of binding nucleotides. In a further embodiment, the NBD domain comprises amino acids 163-457 of SEQ ID NO: 2. In another further embodiment, the NBD domain is encoded by nucleotides 487-1371 of SEQ ID NO: 1 or a degenerate variant thereof.

In another embodiment, the present invention provides an isolated Ipaf polypeptide or functional fragment thereof, wherein the Ipaf polypeptide or functional fragment thereof comprises a LRR domain, said LRR domain having at least 90% identity with amino acids 656-1024 of SEQ ID NO: 2; wherein said LRR domain is capable of interacting specifically with another LRR domain, thereby mediating protein-protein interactions. In a further embodiment, the LRR domain comprises amino acids 656-1024 of SEQ ID NO: 2. In another further embodiment, the LRR domain is encoded by nucleotides 1966-3072 of SEQ ID NO: 1 or a degenerate variant thereof.

In another embodiment, the present invention provides an isolated Ipaf polypeptide or functional fragment thereof, wherein the Ipaf functional fragment comprises amino acids 1-256 of SEQ ID NO: 2, wherein the Ipaf functional fragment induces apoptosis or constitutively induces activation of procaspase-1 or oligomerizes with procaspase-1 or Ipaf of SEQ ID NO: 2. In a further embodiment, the Ipaf functional fragment is encoded by nucleotides 1-768 of SEQ ID NO: 1 or a degenerate variant thereof.

In another embodiment, the present invention provides an isolated Ipaf polypeptide or functional fragment thereof, wherein the Ipaf functional fragment comprises amino acids 1-328 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the Ipaf functional fragment is encoded by nucleotides 1-984 of SEQ ID NO: 1 or a degenerate variant thereof.

In another embodiment, the present invention provides an isolated Ipaf polypeptide or functional fragment thereof, wherein the Ipaf functional fragment comprises amino acids 1-557 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the Ipaf functional fragment is encoded by nucleotides 1-1671 of SEQ ID NO: 1 or a degenerate variant thereof.

In another embodiment, the present invention provides an isolated Ipaf polypeptide or functional fragment thereof, wherein the Ipaf functional fragment comprises amino acids 1-601 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the Ipaf functional fragment is encoded by nucleotides 1-1803 of SEQ ID NO: 1 or a degenerate variant thereof.

In another embodiment, the present invention provides an isolated Ipaf polypeptide or functional fragment thereof, wherein the Ipaf functional fragment comprises amino acids 1-661 of SEQ ID NO: 2, and wherein the Ipaf functional fragment induces apoptosis or constitutively induces activation of procaspase-1 or oligomerizes with procaspase-1 or Ipaf of SEQ ID NO: 2. In a further embodiment, the Ipaf functional fragment is encoded by nucleotides 1-1983 of SEQ ID NO: 1 or a degenerate variant thereof.

In another embodiment, the present invention provides an isolated Ipaf polypeptide or functional fragment thereof, wherein the Ipaf polypeptide comprises at least ten consecutive residues of SEQ ID NO: 2. In yet another embodiment, the present invention provides an isolated Ipaf polypeptide or functional fragment thereof, wherein the Ipaf polypeptide comprises at least 20 consecutive residues of SEQ ID NO: 2.

In another aspect, the present invention provides an antibody that specifically binds an isolated Ipaf polypeptide or functional fragment thereof that has at least 90% amino acid identity with SEQ ID NO: 2, wherein said Ipaf polypeptide or functional fragment thereof induces or suppresses procaspase-1 activation or oligomerizes with procaspase-1. In one embodiment, the antibody has an affinity of at least 10⁻⁷M. In another embodiment, the Ipaf polypeptide is a human Ipaf. In still another embodiment, the Ipaf polypeptide comprises amino acid sequence of SEQ ID NO: 2.

In another embodiment, the present invention provides an antibody that specifically binds a CARD domain of the Ipaf polypeptide or functional fragment thereof, said CARD domain having at least 90% identity with amino acids 1-88 of SEQ ID NO: 2; wherein said CARD domain mediates dimerization between the Ipaf polypeptide or functional fragment thereof or procaspase-1. In a further embodiment, the CARD domain comprises amino acids 1-88 of SEQ ID NO: 2.

In another embodiment, the present invention provides an antibody that specifically binds a NBD domain of the Ipaf polypeptide or functional fragment thereof, said NBD domain having at least 90% identity with amino acids 163-457 of SEQ ID NO: 2, wherein said NBD domain is capable of binding nucleotides. In a further embodiment, the NBD domain comprises amino acids 163-457 of SEQ ID NO: 2.

In another embodiment, the present invention provides an antibody that specifically binds a LRR domain of the Ipaf polypeptide or functional fragment thereof, said LRR domain having at least 90% identity with amino acids 656-1024 of SEQ ID NO: 2; wherein said LRR domain is capable of interacting specifically with another LRR domain, thereby mediating protein-protein interactions. In a further embodiment, the LRR domain comprises amino acids 656-1024 of SEQ ID NO: 2. In certain embodiments, the antibody is a monoclonal, a polyclonal, an antibody fragment, a single chain antibody or a humanized antibody. In addition, cells expressing an Ipaf specific antibody are provided.

In another aspect, the present invention provides a method of identifying an inhibitor or enhancer of an Ipaf mediated caspase processing, wherein the method comprises contacting a sample a sample containing an Ipaf polypeptide or a functional fragment thereof and procaspase-1 or processible fragment thereof with a candidate inhibitor or enhancer, wherein said Ipaf polypeptide or functional fragment thereof has at least 90% amino acid identity with SEQ ID NO: 2, wherein said Ipaf polypeptide or functional fragment thereof induces or suppresses procaspase-1 activation; and detecting the presence of large and small caspase subunits, and therefrom determining the level of caspase processing activity, wherein a decrease in processing indicates the presence of a caspase processing inhibitor, and wherein an increase in processing indicates the presence of a caspase processing enhancer. In one embodiment, the procaspase-1 is in vitro translated and labeled. In another embodiment, the procaspase-1 is labeled, and the label is selected from the group consisting of a radioactive label, a peptide tag, an enzyme and biotin. In an additional embodiment, the step of detection comprises gel electrophoresis.

In another aspect, the present invention provides a method of identifying an inhibitor or enhancer of an Ipaf mediated caspase processing, wherein the method comprises contacting a cell transfected with an expression vector with a candidate inhibitor or enhancer, wherein the expression vector comprises a promoter, and an isolated nucleic acid molecule, wherein the nucleic acid molecule is operably linked to the promoter, and wherein the nucleic acid molecule comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1; and detecting the presence of large and small caspase subunits of procaspase-1, and therefrom determining the level of caspase processing activity, wherein a decrease in processing indicates the presence of a caspase inhibitor, and wherein an increase in processing indicates the presence of caspase processing enhancer. In one embodiment, the step of detection comprises gel electrophoresis.

In another aspect, the present invention provides a method of identifying an inhibitor or enhancer of an Ipaf induced caspase-1 activity, wherein the method comprises contacting a sample containing an Ipaf polypeptide or a functional fragment thereof and procaspase-1 or processible fragment thereof with a candidate inhibitor or enhancer, wherein said Ipaf polypeptide or functional fragment thereof has at least 90% amino acid identity with SEQ ID NO: 2, wherein said Ipaf polypeptide or functional fragment thereof induces or suppresses procaspase-1 activation; and adding caspase-1 substrate into the sample; and detecting the turnover of the substrate, wherein the rate of the turnover of the substrate is indicative of the enzymatic activity of the caspase-1.

In another aspect, the present invention provides a method of identifying an inhibitor or enhancer of an Ipaf mediated apoptosis, wherein the method comprises contacting a cell transfected with an expression vector with a candidate inhibitor or enhancer, wherein the expression vector comprises a promoter, and an isolated nucleic acid molecule, wherein the nucleic acid molecule is operably linked to the promoter, and wherein the nucleic acid molecule comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1; and detecting cell viability, wherein an increase in cell viability indicates the presence of an inhibitor, and wherein an decrease in cell viability indicates an enhancer. In additional aspects, the present invention provides an inhibitor or enhancer identified by any one of the methods disclosed above.

In another aspect, the present invention provides a process for manufacturing an inhibitor or enhancer identified by any one of the methods disclosed above, wherein the process comprises carrying out any one of the methods disclosed above to identify the inhibitor or enhancer, and derivatizing the inhibitor or enhancer. In another aspect, the present invention provides a process of manufacturing a compound, wherein the process comprises providing an inhibitor or enhancer identified by any one of the methods disclosed above, derivatizing the inhibitor or enhancer, and optionally rescreening the inhibitor or enhancer by any one of the methods disclosed above.

In another aspect, the present invention provides a method of inducing apoptosis in a cell, wherein the method comprises delivering to the cell an effective amount of an isolated nucleic acid molecule encoding an Ipaf polypeptide or functional fragment thereof, under conditions and for a time sufficient for expression of the polypeptide and therefrom detecting apoptosis of the cell; wherein said Ipaf polypeptide or functional fragment has at least 90% identity with SEQ ID NO: 2, and wherein said Ipaf polypeptide or functional fragment thereof induces or suppresses procaspase-1 activation. In one embodiment, the step of delivering to the cell is selected from the group consisting of injection, transfection, transformation, electroporation, and receptor mediated endocytosis.

In another aspect, the present invention provides a method of inducing apoptosis in a cell that comprises delivering to the cell an effective amount of an Ipaf polypeptide or functional fragment thereof, under conditions and for a time sufficient to detect therefrom the induction of apoptosis of the cell; wherein said Ipaf polypeptide or functional fragment has at least 90% identity with SEQ ID NO: 2, and wherein said Ipaf polypeptide or functional fragment thereof induces or suppresses procaspase-1 activation. In one embodiment, the Ipaf polypeptide or functional fragment thereof is a CARD domain of the Ipaf polypeptide, said CARD domain comprising amino acids 1-88 of SEQ ID NO: 2. In another embodiment, the step of delivering to the cell in the methods disclosed in this section comprises injecting the polypeptide. In additional embodiments, the step of apoptosis detection in the methods disclosed above uses a technique selected from the group consisting of altered cellular morphology, DNA fragmentation, annexin binding, caspase activity, and mitochondrial release of cytochrome c.

In another aspect, the present invention provides a method of producing an Ipaf polypeptide that comprises culturing a host cell containing an expression vector under conditions and for a time sufficient for expression of the Ipaf polypeptide or functional fragment thereof, wherein the expression vector comprises a nucleic acid molecule encoding the Ipaf polypeptide or functional fragment thereof and a promoter operably linked to the nucleic acid molecule, wherein the nucleic acid molecule comprises a polynucleotide sequence encoding the Ipaf polypeptide or functional fragment thereof, said polypeptide having at least 90% identity with SEQ ID NO: 2, and wherein said Ipaf polypeptide or functional fragment thereof induces or suppresses procaspase-1 activation or oligomerizes with procaspase-1 or Ipaf of SEQ ID NO: 2. In one embodiment, the nucleic acid molecule consists essentially of SEQ ID NO: 1. In additional aspects, the present invention provides an Ipaf polypeptide or functional fragment thereof produced by the method disclosed in this section.

In another aspect, the present invention provides an antisense nucleic acid molecule that comprises a nucleic acid sequence at least 12 nucleotides which is complementary to a nucleic acid molecule encoding an Ipaf polypeptide or functional fragment thereof, wherein the nucleic acid molecule comprises SEQ ID NO: 1, and wherein the Ipaf polypeptide or functional fragment thereof induces or suppresses procasepase-1 activation or oligomerizes with procaspase-1 or Ipaf of SEQ ID NO: 2.

In another aspect, the present invention provides a gene delivery vehicle that comprises a nucleic acid molecule, and a regulatory sequence, wherein the nucleic acid molecule is operably linked to the regulatory sequence; wherein the nucleic acid molecule comprises a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or wherein the polynucleotide sequence has at least 70% identity with SEQ ID NO: 1. In one embodiment, the vehicle is a virus. In a further embodiment, the virus is a retrovirus or adenovirus. In another embodiment, the nucleic acid molecule is associated with a polycation. In an additional embodiment, the vehicle further comprises a ligand that binds a cell surface receptor.

In another aspect, the present invention provides a method of detecting Ipaf transcription levels, wherein the method comprises providing a polynucleotide probe comprising a sequence that specifically hybridizes to an Ipaf polynucleotide sequence or a complement thereof, wherein the Ipaf polynucleotide sequence is presented in SEQ ID NO: 1, contacting a biological sample with the probe under hybridization conditions that permit duplex formation, detecting the presence of the duplex, and comparing detected levels with a control. In one embodiment, the polynucleotide probe comprises at least 15 continuous nucleotides of the nucleic acid sequence presented in SEQ ID NO: 1.

In an additional aspect, the present invention provides a method of detecting Ipaf polypeptide levels, wherein the method comprises providing antibodies that specifically binds to an Ipaf polypeptide or fragment thereof, said Ipaf polypeptide having at least 70% amino acid identity with SEQ ID NO: 2, contacting a biological sample with the antibodies under binding conditions which permit formation of an antibody-polypeptide complex, detecting the presence of said complex; and comparing detected levels with a control.

In another aspect, the present invention provides a kit for screening for agents that alter apoptosis, comprising an isolated nucleic acid molecule comprising a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation; and instructions for use of the same. In an additional aspect, the present invention provides a kit for screening for agents that alter apoptosis, wherein the kit comprises an Ipaf polypeptide or fragment thereof, said Ipaf polypeptide or functional fragment thereof having at least 90% amino acid identity with SEQ ID NO: 2, and a detection reagent that specifically binds to at least one of the foregoing polypeptides. In one embodiment, the detection reagent is an antibody or antigen-binding fragment thereof.

In another aspect, the present invention provides a diagnostic kit comprising a polynucleotide probe comprising a portion of the sequence of SEQ ID NO: 1; wherein the polynucleotide probe specifically hybridizes to an Ipaf nucleic acid molecule or a complement thereof when the Ipaf nucleic acid molecule is present in a test biological sample, wherein the Ipaf nucleic acid molecule encodes an Ipaf polypeptide or functional fragment thereof having at least 70% identity with SEQ ID NO: 2, and instructions for detecting differences between the levels of probe-containing duplexes in the biological sample as compared to a normal biological sample. In an additional aspect, the present invention provides a diagnostic kit comprising an antibody against an Ipaf polypeptide or functional fragment thereof having at least 70% identity amino acid identity with SEQ ID NO: 2, wherein the antibody specifically binds to the Ipaf polypeptide when the polypeptide is present in a test biological sample, and instructions for detecting differences between the levels of antibody-bound polypeptide in the test biological sample and a normal biological sample. In one embodiment, the antibody is a monoclonal antibody, a polyclonal antibody, an antibody fragment, a single chain antibody or a humanized antibody.

In another aspect, the present invention provides a composition that comprises an Ipaf polypeptide or functional fragment thereof, and a physiologically acceptable carrier; wherein said Ipaf polypeptide or functional fragment thereof has at least 90% amino acid identity with SEQ ID NO: 2, and wherein said Ipaf polypeptide or functional fragment thereof induces or suppresses procaspase-1 activation. In one embodiment, the Ipaf polypeptide is a human Ipaf consisting essentially of SEQ ID NO: 2 or functional fragment thereof. In certain further embodiments, the Ipaf polypeptide comprises a CARD domain or a NBD domain or a LRR domain.

In another aspect, the present invention provides a composition that comprises an antibody that selectively binds to a human Ipaf polypeptide having at least 90% amino acid identity with SEQ ID NO: 2 and a physiologically acceptable carrier, wherein said Ipaf polypeptide induces or suppresses procaspase-1 activation. In an additional aspect, the present invention provides a composition that comprises an Ipaf polynucleotide, or fragment or complement thereof, and a physiologically acceptable carrier; wherein said Ipaf polynucleotide encodes an Ipaf polypeptide or a functional fragment thereof, said polypeptide having at least 90% identity with SEQ ID NO: 2; and wherein said functional fragments induce or prevent procaspase-1 activation.

In yet another aspect, the present invention provides a method of treating a mammal with a disease or disorder associated with an Ipaf polypeptide, wherein the method comprises administering to the mammal a composition comprising a therapeutically effective amount of a polynucleotide comprising a sequence capable of binding an Ipaf polynucleotide having at least 70% identity with SEQ ID NO: 2, or a complement thereof. In one embodiment, the polynucleotide is an antisense construct. In another embodiment, the polynucleotide is a ribozyme construct. In yet another embodiment, the polynucleotide comprises a nucleic acid sequence presented in SEQ ID NO: 1, or a complement thereof.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 is the amino acid sequence of human Ipaf (SEQ ID NO:2). The N-terminal CARD domain is highlighted, the internal NBD domain underlined, the Walker P-loop shadowed and the C-terminal LRR domain boxed.

FIG. 2 is the schematic diagram representing the domain structure of Ipaf.

FIG. 3 is the amino acid sequence alignment of the CARD domain of Ipaf (SEQ ID NO: 3) with CARD domains found in human Apaf-1(SEQ ID NO:4), Nod1 (SEQ ID NO: 5), CED-4 (SEQ ID NO: 6), caspase-1 (SEQ ID NO: 7), caspase-4 (SEQ ID NO: 8), caspase-5 (SEQ ID NO: 9), and caspase-13 (SEQ ID NO: 10).

FIG. 4 is a scanned image of PCR products detecting the expression of Ipaf mRNA in various tissues and cell lines.

FIG. 5 is a fluorescent image of subcellular localization of Ipaf in transiently transfected MCF7 cells. MCF7 cells were transfected with GFP-tagged Ipaf full length or a CARD-truncated mutant (residues 80-1024). 24 hours following transfection, cells were examined by confocal fluorescent microscopy and photographed.

FIG. 6 is a scanned immunoblot showing self-oligomerizatin of Ipaf through its CARD domain. 293T cells were cotransfected with T7-Ipaf together with one of the following constructs: empty vector (Vector), Flag-tagged full length Ipaf (FL of SEQ ID NO: 2), Flag-tagged CARD domain (residues 1-88 of SEQ ID NO: 2) of Ipaf (CARD), or Flag-tagged CARD-deleted mutant (residues 80-1024 of SEQ ID NO: 2) of Ipaf (ΔCARD). 24 hours following transfection, cells were lysed and the lysates immunoprecipitated (IP) with anti-Flag antibody. The immunoprecipitates were immunoblotted (IB) with anti-T7 antibody. Expression of T7-Ipaf or Flag-tagged constructs was determined by immunoblotting (IB) with anti-T7 or anti-Flag antibodies, respectively.

FIG. 7 is a scanned autoradiography showing in vitro interaction of Ipaf with its CARD domain. ³⁵S-labeled Ipaf (lane 1, 10% input) was incubated with an equal amount of glutathione Sepharose beads bound to an equal amount of GST (lane 2) or Ipaf-CARD-GST fusion protein (lane 3). Bound proteins were then eluted and analyzed by SDS-PAGE and autoradiography.

FIG. 8 is a scanned immunoblot showing the in vivo interaction between Ipaf and procaspase-1 through their CARD domains. 293T cells were cotransfected with either empty vector or Flag-tagged full length (Flag-Ipaf or FL of SEQ ID NO: 2), CARD domain of residues 1-88 of SEQ ID NO: 2 (Flag-CARD Ipaf or CARD) or a CARD domain-deleted mutant of residues 80-1024 of SEQ ID NO: 2 (Flag-ΔCARD Ipag or ΔCARD) of Ipaf together with either “empty” vector or T7-tagged full length (T7-procaspase-1) or CARD domain of residues 1-94 (T7-CARD caspase-1) of procaspase-1 expression constructs. 24 hours following transfection, cells were lysed and the lysates immunoprecipitated with anti-Flag antibody. The immunoprecipitates were immunoblotted with anti-T7 antibody. The expression of different T7- or Flag-tagged chimeras was determined by immunoblotting with anti-Flag or anti-T7 antibodies, respectively.

FIG. 9 is a scanned autoradiograph showing in vitro interaction of procaspase-1 with the CARD domain of Ipaf. ³⁵S-labeled procaspase-1 (lane 1, 10% input) was incubated with an equal amount of glutathione Sepharose beads bound to an equal amount of GST (lane 2) or Ipaf-CARD-GST (lane 3). Bound proteins were then eluted and analyzed by SDS-PAGE and autoradiography.

FIG. 10 is a scanned immunoblot showing the in vivo processing of procaspase-1 by Ipaf. 293T cells were cotransfected with procaspase-1 expression construct (Flag-procaspase-1) together with either vector control, RICK or Ipaf 1-661 constructs. 18 hours following transfection, the N-terminally Flag-tagged proform and mature caspase-1 in the total lysates were detected by anti-Flag antibody.

FIG. 11 is a graph illustrating the potentiation of caspase-1-induced cell death by Ipaf. 293T cells were transfected with procaspase-1 plasmid plus β-galactosidase plasmid and either empty vector or the indicated Ipaf constructs together with or without an active site mutant (C285A) procaspase-1 expression construct. 20 hours following transfection, cells were fixed and the morphology of 5-bromo-4-chloro-3-indolyl β-D-galactopyranoside-stained cells examined by light microscopy. Data (mean +/− S.E.) shown are the percentage of apoptotic cells among the total number of cells counted (n=3).

FIG. 12 is a scanned immunoblot showing the in vivo processing of procaspase-1 by Ipaf. 293T cells were cotransfected with Flag-tagged procaspase-1 expression construct (Flag-procaspase-1) in the presence of either vector control, RICK or the indicated Ipaf constructs. 18 hours following transfection, the N-terminally Flag-tagged proform and mature caspase-1 polypeptides in the total lysates were detected by anti-Flag antibody.

FIG. 13 is a scanned immunoblot showing that mutation of cysteine 285 of procaspase-1 abolishes its processing by Ipaf. 293T cells were cotransfected with an active site mutant (C285A) procaspase-1 expression construct (T7-procaspase-1 C285A) in the presence of either vector control, RICK or Ipaf constructs (i.e., Ipaf 1-601). 18 hours following transfection, processing of the T7-tagged C285A caspase-1 in the total lysates was probed by anti-T7 antibody.

### DETAILED DESCRIPTION OF THE INVENTION

Prior to setting forth the invention, it may be helpful to an understanding thereof to set forth definitions of certain terms that will be used hereinafter.

An “isolated nucleic acid molecule” refers to a polynucleotide molecule in the form of a separate fragment or as a component of a larger nucleic acid construct, that has been separated from its source cell (including the chromosome it normally resides in) at least once, and preferably in a substantially pure form. Nucleic acid molecules may be comprised of a wide variety of nucleotides, including DNA, RNA, nucleotide analogues, or a combination thereof.

Within the context of the present invention, “antibodies” are understood to include monoclonal antibodies, polyclonal antibodies, anti-idiotypic antibodies, and antibody fragments (e.g., Fab, and F(ab′)₂, Fᵥ variable regions, or complementary determining regions).

A “functional fragment”, as used herein, refers to a fragment derived from the full length Ipaf polypeptide that retains at least one functional activity associated with full-length Ipaf including induction or suppression of procaspase-1 activation or oligomerization with procaspase-1 or Ipaf of SEQ ID NO: 2. Such functional fragments may include, for example, the CARD domain, the NBD domain and/or the LRR domain.

References to “Ipaf” herein is intended to include a polypeptide of any origin which is substantially homologous to and which is biologically equivalent to the Ipaf polypeptides characterized and described herein.

A. Ipaf Nucleic Acid Molecules

The present invention provides nucleic acid molecules that encode Ipaf, or fragments or variants thereof. The invention discloses the nucleic acid sequences of human Ipaf (SEQ ID NO:1 and SEQ ID NO:14). However, the invention is not limited to these specific nucleic acid sequences. The invention includes any and all nucleic acid sequences that encode an Ipaf polypeptide or fragment or variant thereof. Ipaf nucleic acids derived from any species are also within the scope of the invention.

The human Ipaf nucleic acid molecules of the present invention were identified by searching sequence databases with the CARD domain sequence of procaspase-1 as described in Example 1 and have been submitted to the GenBank/EBI Data Bank with accession number AY035391. The nucleic acid sequences for the coding region of human Ipaf nucleic acid molecules are provided in SEQ ID NO: 1 and a full length human Ipaf cDNA sequence is provided in SEQ ID NO:14.

Ipaf nucleic acid molecules may be isolated from genomic DNAs or cDNAs according to practices known in the art (see Sambrook et al., Molecular Cloning: A Laboratory Manual, Cold Spring Harbor Press, 1989). Nucleic acid probes corresponding to a region of the Ipaf sequences disclosed in the invention may be used to screen either genomic or cDNA libraries. An oligonucleotide probe suitable for screening genomic or cDNA libraries is generally 20-40 bases in length. The oligonucleotide may be synthesized or purchased commercially. The oligonucleotide may be labeled with a variety of molecules which facilitate detection, such as a radionuclide (e.g., ³²P), an enzymatic label, a protein label, a fluorescent label or biotin.

Genomic and cDNA libraries may be constructed in a variety of suitable vectors including, for example, plasmid, bacteriophage, yeast artificial chromosome and cosmid vectors. Alternatively, libraries may be purchased from commercial sources (e.g., Clontech, Palo Alto, Calif.). Libraries may contain genomic DNA or cDNA inserts isolated from any species. Nucleotide probes corresponding to the human Ipaf sequences disclosed in the present invention can be used to screen libraries constructed from DNA isolated from other species and, therefore, to identify and isolate other Ipaf nucleic acid molecules within the scope of the present invention.

Other methods may also be utilized to obtain Ipaf nucleic acid molecules. One preferred method is to perform polymerase chain reaction (PCR) to amplify an Ipaf nucleic acid molecule from cDNA or genomic DNA using oligonucleotide primers corresponding to the 5′ and 3′ ends of Ipaf nucleic acid molecules or regions thereof. Detailed methods of PCR cloning may be found in Ausubel et al., Current Protocols in Molecular Biology, Greene Publishing Associates and Wiley-Interscience, NY, 1995, for example. The full length human nucleic acid molecule can be isolated by PCR using human genomic template and PCR adaptor-primers which encompass the start and stop sites of the Ipaf open reading frame.

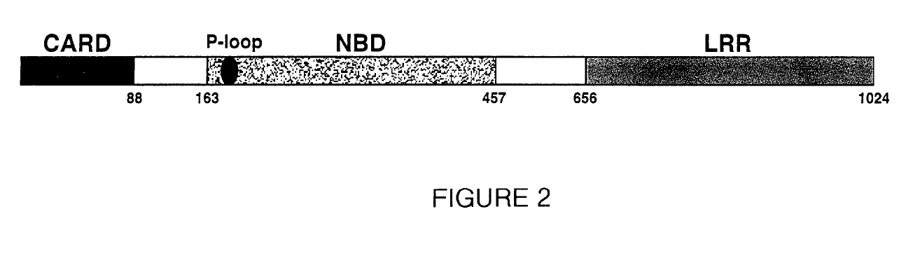

Another preferred method of obtaining an Ipaf nucleic acid molecules is by expression cloning using a polypeptide probe capable of binding an Ipaf polypeptide. The probe may comprise an anti-Ipaf antibody, an Ipaf binding partner, or, alternatively, an Ipaf polypeptide containing a dimerization domain, for example. Methods of expression cloning are described in Sambrook et al., Molecular Cloning: A Laboratory Manual, Cold Spring Harbor Press, 1989; Ausubel et al., Current Protocols in Molecular Biology, Greene Publishing Associates and Wiley-Interscience, NY, 1995; and Blackwood and Eisenman, Methods in Enzymology 254:229-40, 1995. Antibody probes suitable for cross-species cloning can include those directed against conserved regions of Ipaf polypeptides such as the CARD or NBD or LRR domains, for example. Preferably, the antibodies will bind to the denatured Ipaf polypeptide. Preferred Ipaf polypeptide probes include those corresponding to a CARD domain, since this domain mediates dimerization between Ipaf proteins. An Ipaf CARD domain probe is particularly well suited to cross-species cloning, since CARD domains of human Ipaf are capable of dimerization (see FIG. 2(A)).

Ipaf nucleic acid molecules may also be isolated by yeast two-hybrid cloning using the CARD domain of an Ipaf polypeptide as the bait. Methods of yeast two-hybrid cloning have been described and are widely used in the art (see, e.g., Gyuris et al., Cell 75:791-803, 1993). Yeast two-hybrid vectors and libraries are commercially available (e.g., Matchmaker Systems™, Clontech, Palo Alto, Calif.).

Polynucleotides of the invention may also be made using the techniques of synthetic chemistry given the sequences disclosed herein. The degeneracy of the genetic code permits alternate nucleotide sequences which will encode the amino acid sequences presented in SEQ ID NO: 2. All such nucleotide sequences are within the scope of the present invention.

Isolated genes corresponding to the cDNA sequences disclosed herein are also provided. Methods such as those described above can be used to isolate genes (genomic clones) which correspond to known cDNA sequences. Preferred methods include screening genomic libraries with probes comprising cDNA fragments and PCR amplification of genomic clones from genomic libraries. All polypeptides encoded by the isolated genes are within the scope of the invention. These polypeptides include, but not limited to, polypeptides encoded by the cDNAs comprising SEQ ID NO:1, isoforms of these polypeptides resulting from alternative splicing of the isolated genes, as well as functional fragments thereof.

Ipaf nucleic acid molecules from a variety of species may be isolated using the compositions provided herein. For closely related species, the human sequence or portion thereof may be utilized as a probe to screen a genomic or cDNA library. For example, a fragment of a polynucleotide that encodes a polypeptide region that encompasses the CARD domain of Ipaf may be labeled and used as a probe on a library constructed from mouse, primate, rat, dog, or other vertebrate, warm-blooded or mammalian species. An initial hybridization at moderate stringency may yield candidate clones or fragments. If no hybridization is initially observed, varying degrees of stringency may be used (see Sambrook et al., supra, and other well-known sources for stringent conditions). While such probes may also be used to probe libraries from evolutionarily diverse species, such as Drosophila, hybridization conditions will likely be less stringent.

While relaxed hybridization conditions using probes designed from human sequences may identify Ipaf nucleic acid molecules of evolutionarily diverse species, it may be more beneficial to attempt to directly isolate these molecules from a library using methods which do not require the human sequence per se. These methods include, but are not limited to, amplification using primers derived from conserved areas, amplification using degenerate primers from various regions, antibody probing of expression libraries, and the like. For example, random-primed amplification (e.g., polymerase chain reaction) may be employed (see, e.g., Methods Enzymol. 254:275, 1995; Trends Genet. 11:242, 1995; Liang and Pardee, Science 257:967, 1992; Welsh et al., Nucl. Acids Res. 20:4965, 1992). In addition, variations of random-primed PCR may also be used, especially when a particular gene or gene family is desired. In such a method, one of the amplification primers is an “anchored oligo(dT) (oligo(dT)dN)” and the other primer is a degenerate primer based upon amino acid or nucleotide sequence of a related gene. A gene sequence is identified as an Ipaf by amino acid similarity and/or nucleic acid similarity. Generally, amino acid similarity is preferred. A gene sequence may also be identified as an Ipaf if it encodes a polypeptide with sequence similarity to an Ipaf polypeptide or with functional characteristics of Ipaf or a functional domain of Ipaf. Candidate Ipaf genes may be examined for enzyme activity by one of the functional assays described herein, or other equivalent assays. Candidate Ipaf genes may also be identified by methods of expression screening or yeast two-hybrid screening using a probe expressing a functional domain capable of binding to an Apaf polypeptide, for example.

In certain embodiments, compositions and methods of the invention include ribozymes, antisense RNA, dsRNA, siRNA, shRNA, ssDNA, and dominant-negative Ipaf mutants to decrease levels of functional Ipaf polypeptides in a cell. Ribozymes are trans-cleaving catalytic RNA molecules possessing endoribonuclease activity. Ribozymes are specifically designed for a particular target nucleotide sequence. Ribozymes are engineered to cleave an RNA species site-specifically in the background of cellular RNA. The cleavage event renders the mRNA unstable and prevents protein expression. Preparation and usage of ribozymes is well known to the art (see, Usman et al., Current Opin. Struct. Biol. 6:527-533, 1996; Long et al., FASEB J. 7:25, 1993; Symons, Ann. Rev. Biochem. 61:641, 1992 and U.S. Pat. No. 5,254,678). The Ipaf nucleic acid sequences provided by the invention allow construction of an effective Ipaf ribozyme.

The complements of the nucleotide sequences shown in SEQ ID NO:1 and SEQ ID NO:14 are contiguous nucleotide sequences which form Watson-Crick base pairs with a contiguous nucleotide sequence as shown in SEQ ID NO:1 or SEQ ID NO:14. The complements of the nucleotide sequences shown in SEQ ID NO:1 and SEQ ID NO:14 (the antisense strand) may be used to provide antisense oligonucleotides.

Antisense nucleic acids are designed to specifically bind to RNA, resulting in the formation of RNA-DNA or RNA-RNA hybrids and an arrest in DNA replication, reverse transcription or messenger RNA translation. Antisense polynucleotides based on a selected sequence can specifically interfere with expression of the corresponding gene. Antisense polynucleotides are typically generated within the cell by expression from antisense constructs that contain the antisense strand as the transcribed strand. Antisense production and uses thereof are discussed extensively in the literature and are widely known and available to one skilled in the art. (see Agrwal et al., Tet. Lett. 28:3539-3542, 1987; Miller et al., J. Am. Chem. Soc. 93:6657-6665, 1971; Stec et al., Tet. Lett. 26:2191-2194, 1985; Moody et al., Nucl. Acids Res. 12:4769-4782, 1989; Eckstein, Trends Biol. Sci. 14:97-100, 1989; Stein In: Oligodeoxynucleotides: Antisense Inhibitors of Gene Expression, Cohen, Ed, Macmillan Press, London, pp. 97-117, 1989; U.S. Pat. Nos. 5,168,053; 5,190,931; 5,135,917; 5,087,617; and 5,176,996). Effective Ipaf antisense expression vectors may be produced based on the Ipaf nucleic acid sequences provided by the invention, but typically will include at least 10 contiguous nucleotides. In certain embodiments no more than 100 contiguous nucleotides of Ipaf are used, while in other embodiments between 18-60 or between 20-50 (including all integer values in-between) contiguous nucleotides of Ipaf are utilized.

The procedure of double-stranded RNA interference (RNAi) may also be used to specifically inhibit expression of an associated gene, such as an Ipaf gene. Briefly, the presence of double-stranded RNA dominantly silences gene expression in a sequence-specific manner by causing the corresponding endogenous RNA to be degraded. Although first discovered in lower organisms such as the nematode and Drosophila, for example, dsRNAi has been demonstrated to work in mammalian cells (Wianny, F. and Zernica-Goetz, M., (2000), NATURE CELL BIOLOGY Vol 2., 70-75. The mechanism behind RNA interference is still not entirely understood, but it appears that a double-stranded RNA (dsRNA) is broken into short pieces, typically 21-23 nucleotides in length, termed short interfering RNAs (siRNA). The siRNA triggers the degradation of mRNA that matches its sequence, thereby repressing expression of the corresponding gene. Discussed in Bass, B., NATURE 411:428-429 (2001) and Sharp, P. A., GENES DEV. 15:485-490 (2001). Indeed, the introduction of siRNAs to a cell can trigger RNAi in mammalian cells (Elbashir, S. M., et al. NATURE 411:494-498 (2001)), and the invention therefore contemplates the use of siRNAs to target degradation of mRNA encoding an Ipaf polypeptide.

Shagging RNAs may also be used to inhibit or decrease Ipaf expression, according to the invention. Shagging RNA (shRNA) is a form of hairpin RNA capable of sequence-specifically reducing expression of a target gene. A recent study established that such short hairpin activated gene silencing (which the researchers termed “SHAGging”) in a variety of normal and cancer cell lines, and in mouse cells as well as in human cells. Paddison, P. et al., GENES DEV. 16(8):948-58 (2002).

Single-stranded DNA fragments (ssDNA) may also be used to inhibit or decrease Ipaf expression. In specific embodiments, triplex molecules may be used to inhibit gene expression via a gene expression inhibitor element. Triplex molecules refer to single DNA strands that bind duplex DNA, thereby forming a collinear triplex molecule and preventing transcription (see, e.g., U.S. Pat. No. 5,176,996 to Hogan et al., which describes methods for making synthetic oligonucleotides that bind to target sites on duplex DNA).

In certain embodiments, dominant negative Ipaf mutants may be generated by deleting or substituting a portion or one or more nucleotides of the Ipaf coding sequence. The expression “dominant negative,” as used herein, refers to certain Ipaf mutants that can suppress at least one of the functions of normal Ipaf polypeptides in a cell when the mutant is expressed in the same cell. Without intending to be bound by any particular theory or mechanism, it is assumed that the dominant negative Ipaf mutants may interact or associate with normal Ipaf polypeptides or binding partners inside of a cell in such a way that the interaction or association sequesters or prevents the normal Ipaf polypeptides from functioning properly. For example, the dominant negative Ipaf mutants may interact or associate with natural substrates or targets of the normal Ipaf polypeptides and prevent the normal Ipaf polypeptide from effecting upon the normal substrates or targets. It is contemplated that the dominant negative Ipaf mutants may be used in many situations. For example, dominant negative Ipaf mutants may attenuate Ipaf-induced apoptosis without changing the levels of normal Ipaf polypeptides in a cell. The dominant negative Ipaf mutants include, for example, a CARD domain-less Ipaf polypeptide. Any nucleic acid sequence that encodes a dominant negative Ipaf mutant that diminishes or blocks any Ipaf function or binding interaction is included in the present invention.

In certain embodiments, constitutively active Ipaf mutants or functional fragments may be generated by deleting or substituting a portion or one or more nucleotides of the Ipaf coding sequence. The expression “constitutively active,” as used herein, refers to Ipaf mutants or functional fragments that demonstrate at least one of the functions of an activated Ipaf polypeptide. Without being bound by any particular theory or mechanism, it is assumed that the Ipaf polypeptide contains a negatively regulatory sequence that may restrain, inhibit, or prevent the Ipaf polypeptide from being active without external activation. It is reasoned that a removal of the negatively regulatory sequence from the Ipaf polypeptide may negate the necessity of external activation so that the mutant becomes constitutively active. It is contemplated that the constitutively active Ipaf mutants may be applicable in many situations, such as inducing apoptosis and screening inhibitors or enhancers of Ipaf-induced procaspase-1 activation. The constitutively active Ipaf mutants include, for example, an LRR domain-less Ipaf polypeptide. Any nucleic acid sequence encoding a constitutively active Ipaf polypeptide regarding any function or binding of Ipaf is included in the present invention. Constitutively active Ipaf polypeptides include any polypeptide with any level or degree of Ipaf activity, even if this activity is less than or more than the level of activity associated with a native active Ipaf polypeptide.

Nucleic acid sequences encoding Ipaf polypeptides may be fused to a variety of sequences including those encoding a secretion signal, whereby the resulting polypeptide is a precursor protein which is subsequently processed and secreted, and an epitope or functional tag sequence. The resulting processed Ipaf polypeptide may be recovered from the cell lysate, periplasmic space, phloem, or from the growth or fermentation medium. Secretion signals suitable for use are widely available are well known in the art (e.g., von Heijne, J. Mol. Biol. 184:99-105, 1985).

The Ipaf nucleic acid molecules of the present invention also include variants (including alleles) of the native nucleic acid molecules identified in SEQ ID NO:1, SEQ ID NO:14, and fragments of either. Variants of the Ipaf nucleic acid molecules provided herein include natural variants (e.g., polymorphisms, splice variants or mutants) and those produced by genetic engineering. Many methods for generating mutants have been developed (see generally, Ausubel et al., supra). Preferred methods include alanine scanning mutagenesis and PCR generation of mutants using an oligonucleotide containing the desired mutation to amplify mutant nucleic acid molecules. Variants generally have at least 70% or 75% nucleotide identity to the native sequence, preferably at least 80%-85%, and most preferably at least 90%, 95%, or 98% nucleotide identity with SEQ ID NO:1 or SEQ ID NO:14. Variants may have any integer value of percent nucleotide homology within the ranges set forth above to the native sequence. The identity algorithms and settings that may be used are defined herein infra, but may also include using computer programs which employ the Smith-Waterman algorithm, such as the MPSRCH program (Oxford Molecular), using an affine gap search with the following parameters: a gap open penalty of 12 and a gap extension penalty of 1. A preferred method of sequence alignment uses the GCG PileUp program (Genetics Computer Group, Madison, Wis.) (Gapweight: 4, Gaplength weight: 1). Other preferred algorithms are the BLAST algorithms using default parameters. Further, a nucleotide variant will typically be sufficiently similar in sequence to hybridize to the reference sequence under moderate or stringent hybridization conditions. For nucleic acid molecules over about 500 bp, stringent conditions include a solution comprising about 1 M Na⁺ at 250 to 30° C. below the Tm; e.g., 5× SSPE, 0.5% SDS, at 65° C.; see Ausubel et al., Current Protocols in Molecular Biology, Greene Publishing, 1995; Sambrook et al., Molecular Cloning: A Laboratory Manual, Cold Spring Harbor Press, 1989). Typically, homologous polynucleotide sequences can be confirmed by hybridization under stringent conditions, as is known in the art. For example, using the following wash conditions: 2× SSC (0.3 M NaCl, 0.03 M sodium citrate, pH 7.0), 0.1% SDS, room temperature twice, 30 minutes each; then 2× SSC, 0.1% SDS, 50° C. once, 30 minutes; then 2× SSC, room temperature twice, 10 minutes each, homologous sequences can be identified which contain at most about 25-30% basepair mismatches. More preferably, homologous nucleic acid strands contain at most 15-25% basepair mismatches, even more preferably at most 5-15% basepair mismatches.

Typically, for stringent hybridization conditions a combination of temperature and salt concentration should be chosen that is approximately 12-20° C. below the calculated Tm of the hybrid under study. The Tm of a hybrid between a nucleotide sequence as shown in SEQ ID NO:1 and a polynucleotide sequence which is 70%, 75%, 80%, 85%, 90%, 95%, 96%, 97%, 98%, or 99% identical can be calculated, for example, using the equation of Bolton and McCarthy, Proc. Natl. Acad. Sci. U.S.A. 48, 1390, 1962:

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4× SSC at 65° C., or 50% formamide, 4× SSC at 42° C., or 0.5× SSC, 0.1% SDS at 65° C. Highly stringent wash conditions include, for example, 0.2× SSC at 65° C. Suitable moderately stringent conditions include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2×SSC containing 0.1% SDS.

Variants of Ipaf nucleic acid molecules provided herein may be engineered from natural variants (e.g., polymorphisms, splice variants, mutants), synthesized or constructed. Many methods have been developed for generating mutants (see, generally, Sambrook et al., supra; Ausubel, et al., supra, and the discussion above). Briefly, preferred methods for generating nucleotide substitutions utilize an oligonucleotide that spans the base or bases to be mutated and contains the mutated base or bases. The oligonucleotide is hybridized to complementary single stranded nucleic acid and second strand synthesis is primed from the oligonucleotide. The double-stranded nucleic acid is prepared for transformation into host cells, typically E. coli, but alternatively, other prokaryotes, yeast or other eukaryotes. Standard screening and vector growth protocols are used to identify mutant sequences and obtain high yields.

Similarly, deletions and/or insertions of the Ipaf nucleic acid molecule may be constructed by any of a variety of known methods as discussed supra. For example, the nucleic acid molecule can be digested with restriction enzymes and religated, thereby deleting or religating a sequence with additional sequences, such that an insertion or large substitution is made. Other means of generating variant sequences may be employed using methods known in the art, for example those described in Sambrook et al., supra; Ausubel et al., supra. Verification of variant sequences is typically accomplished by restriction enzyme mapping, sequence analysis, or probe hybridization.

Furthermore, the term “variant” when used in the context of a polynucleotide sequence, may encompass a polynucleotide sequence related to SEQ ID NO: 1. This definition may include, for example “allelic” (as defined above), “splice,” “species,” “polymorphic,” or “degenerate” variants. A splice variant may have significant identity to a reference molecule, but will generally have a greater of lesser number of polynucleotides due to alternate splicing of exons during mRNA processing. The corresponding polypeptide may possess additional functional domains or an absence of one or more domains, for example. Species variants are polynucleotide sequences that vary from one species to another. The resulting polypeptides generally will have significant amino acid identity to each other. A polymorphic variant is a variation in the polynucleotide sequence of a particular gene between individuals within a given species. Polymorphic variants may also encompass “single nucleotide polymorphisms” (SNPs) in which the polynucleotide sequence varies by one base. The presence of SNPs may be indicative of, for example, a certain population, a disease state, or a propensity for a disease state. A degenerate variant encompasses a multitude of polynucleotides that encode SEQ ID NO: 2. The degenerate variants may occur naturally or may be produced synthetically. Synthetic degenerate variants are made in accordance with the standard triplet genetic code as applied to the polynucleotide sequence of naturally occurring Ipaf polypeptide, and all such variations are to be considered as being specifically disclosed.

Nucleic acid sequences which are substantially the same as the nucleic acid sequences encoding Ipaf are included within the scope of the invention. Such substantially same sequences may, for example, be substituted with codons optimized for expression in a given host cell such as E. coli. The present invention also includes nucleic acid sequences which will hybridize to sequences which encode human Ipaf or complements thereof. The invention includes nucleic acid sequences encoding functional domains of Ipaf proteins, such as, for example, CARD domains and NBD domains. Deletions, insertions and/or nucleotide substitutions within an Ipaf nucleic acid molecule are also within the scope of the current invention. Such alterations may be introduced by standard methods known in the art such as those described in Ausubel et al., supra. In addition, the invention includes nucleic acids that encode polypeptides that are recognized by antibodies that bind an Ipaf polypeptide or fragment thereof or a polypeptide sharing functional activity of Ipaf or a fragment thereof.

Genes that encode the Ipaf polypeptides of the present invention may have the coding sequences shown in SEQ ID NO:1. Polynucleotide molecules of the invention contain less than a whole chromosome and can be single- or double-stranded. Preferably, the polynucleotide molecules are intron-free. Polynucleotide molecules of the invention can comprise at least 11, 15, 18, 21, 30, 33, 42, 54, 60, 66, 72, 84, 90, 100, 120, 140, 160, 180, 200, 220, 240, 250, 260, 300, 330, 400, 420, 500, 540, 600, 660, 700, or more (including all integer values therebetween) contiguous nucleotides selected from nucleotides of SEQ ID NO:1, SEQ ID NO:14, or the complements of either.

Polynucleotide molecules of the invention also include molecules which encode single-chain antibodies which specifically bind to the disclosed proteins, ribozymes which specifically bind to mRNA encoding the disclosed proteins, and fusion proteins comprising amino acid sequences of the disclosed proteins.

B. Ipaf Polypeptides

The present invention includes polypeptide sequences corresponding to Ipaf. The invention discloses the amino acid sequences of human Ipaf represented in SEQ ID NO:2. Such substantially homologous polypeptides may be native to any species, and their biological activity can be determined by any suitable functional assay. For example, biological equivalents may be identified by examining their effect on apoptosis or procaspase-1 processing. Alternatively, biological equivalency can be established by the ability to bind to an Ipaf domain containing polypeptide. The term “biologically equivalent” is intended to mean that the compositions of the present invention are capable of demonstrating one, some or all of the same biological properties, although not necessarily to the same degree, as the Ipaf polypeptides described herein or recombinantly produced Ipaf of the invention. The term “substantially homologous” is intended to mean that the degree of homology between human Ipaf to an Ipaf from any species is greater than that between Ipaf and any other previously reported CARD domain containing protein.

The current invention encompasses all variants (including alleles) of the native Ipaf polypeptide sequence. Such variants may result from natural polymorphisms or may be synthesized by recombinant methodology, and differ from wild-type polypeptides by one or more amino acid substitutions, insertions, deletions, or the like. Amino acid changes in variants or derivatives of Ipaf proteins may be conservative substitutions. Guidance in determining which amino acid residues can be substituted, inserted, or deleted without abolishing biological or immunological activity can be found using computer programs well known in the art, such as DNASTAR software. Preferably, amino acid changes in secreted protein variants are conservative amino acid changes, i.e., substitutions of similarly charged or uncharged amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Whether an amino acid change results in a functional secreted protein or polypeptide can readily be determined by testing the altered protein or polypeptide in functional assays disclosed in the present invention.

A conservative amino acid change involves substitution of one amino acid for another amino acid of a family of amino acids with structurally related side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified as aromatic amino acids. Non-naturally occurring amino acids can also be used to form protein variants of the invention.

A “functionally conservative mutation” as used herein intends a change in a polynucleotide encoding a derivative polypeptide in which the activity is not substantially altered compared to that of the polypeptide from which the derivative is made. Such derivatives may have, for example, amino acid insertions, deletions, or substitutions in the relevant molecule that do not substantially affect its properties. For example, the derivative can include conservative amino acid substitutions, such as substitutions which preserve the general charge, hydrophobicity/hydrophilicity, side chain moiety, and/or stearic bulk of the amino acid substituted, for example, Gly/Ala, Val/Ile/Leu, Asp/Glu, Lys/Arg, Asn/Gln, Thr/Ser, and Phe/Trp/Tyr.

In the region of homology to the native sequence represented by SEQ ID NO: 2, variants should preferably have at least 70%, 75%, 80%, or 90% amino acid identity, and within certain embodiments, at least 90%, 91%, 92%, 93%, 94%, 95%, or 97% identity. In certain embodiments the full length sequence is compared to a test sequence or when necessary a particular domain is compared to a test sequence to determine percent identity. Such amino acid sequence identity may be determined by standard methodologies, including those set forth supra as well as the use of the National Center for Biotechnology Information BLAST 2.0 search methodology (Altschul et al., J. Mol. Biol. 215:403-10, 1990). In one embodiment BLAST 2.0 is utilized with default parameters. A preferred method of sequence alignment uses the GCG PileUp program (Genetics Computer Group, Madison, Wis.) (Gapweight: 4, Gaplength weight: 1). The pileUp program creates a multiple sequence alignment from a group of related sequences using progressive, pairwise alignments. PileUp creates a multiple sequence alignment using the progressive alignment method of Feng and Doolittle (J. Mol. Evol. 25:351-360, 1987) and is similar to the method described by Higgins and Sharp (CABIOS 5:151-153, 1989). Further, whether an amino acid change results in a functional protein or polypeptide can be readily determined by assaying biological properties of the disclosed proteins or polypeptides. For example, the biological properties of Ipaf variants can be assayed by examining their effects on apoptosis and/or procaspase-1 processing, as described in Examples 12-13. Additionally, an Ipaf polypeptide can be functionally assayed by its ability to bind a CARD domain.

The present invention also provides a functional fragment of an Ipaf polypeptide. Such a functional fragment is defined structurally and functionally in that it has amino acid sequence identity to a portion of SEQ ID NO: 2, as described below, or has at least one biological activity of Ipaf, as described above. Functional fragments may exhibit decreased or increased biological activity as compared to native or full length Ipaf. In one embodiment, such a functional fragment comprises at least 10 contiguous amino acids and has at least about 70% amino acid sequence identity with a portion of SEQ ID NO:2. In other embodiments, such a functional fragment comprises at least 10 contiguous amino acids and has at least about 75% or 80% amino acid sequence identity with a portion of SEQ ID NO:2. In other embodiments, such a functional fragment comprises at least 10 contiguous amino acids and has at least about 85% or 90% amino acid sequence identity with a portion of SEQ ID NO:2. In another embodiment, such a functional fragment comprises at least 25 contiguous amino acids and has at least about 65% amino acid sequence identity with a portion of SEQ ID NO:2. In other embodiments, such a functional fragment comprises at least 25 contiguous amino acids and has at least about 70% or 75% amino acid sequence identity with a portion of SEQ ID NO:2. In other embodiments, such a functional fragment comprises at least 25 contiguous amino acids and has at least about 80% or 85% amino acid sequence identity with a portion of SEQ ID NO:2. In other embodiments, such a functional fragment comprises at least 40 contiguous amino acids and has at least about 50% or 60% amino acid sequence identity with a portion of SEQ ID NO:2. In yet other embodiments, such a functional fragment comprises at least 40 contiguous amino acids and has at least about 70% or 80% amino acid sequence identity with a portion of SEQ ID NO:2. The aforementioned identities may be calculated with any one of the algorithms herein described.

The present invention also encompasses the use of mutant and variant forms of genes or polypeptides, including dominant negative mutants. Dominant negative mutations are readily generated for a variety of proteins, including those which are active in homo- or heteromeric complexes. A mutant polypeptide may interact with wild-type binding partners and form a non-functional multimer or a multimer with altered, decreased or enhanced function. Thus, a preferred mutation is in a binding domain, a catalytic domain, or a cellular localization domain. Preferred Ipaf dominant-negative mutants include the CARD domain, the NBD domain and the LRR domain. Preferably, the dominant-negative polypeptide will be overproduced compared to wild type expression. Point mutations and deletions may be constructed which have such an effect. In addition, fusion of different polypeptides of various lengths to the terminus of a protein, such as Ipaf, can yield dominant negative mutants (see Herskowitz, Nature 329:219-222, 1987).

Ipaf variants also include hybrid and modified forms of Ipaf polypeptides such as, but not limited to, fusion proteins and Ipaf fragments. Ipaf fusion proteins include polypeptides comprising Ipaf or a fragment thereof fused to amino acid sequences comprising one or more heterologous polypeptides. Such heterologous polypeptides may correspond to naturally occurring polypeptides of any source or may be recombinantly engineered amino acid sequences. Fusion proteins are useful for purification, generating antibodies against amino acid sequences, and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of one or more of the disclosed proteins can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can be selected from any contiguous amino acid sequences shown in SEQ ID NO:2 as herein described or from biologically active variants of those sequences. The first protein segment can consist of a full-length Ipaf protein.

Other first protein segments can consist of at least 8, 10, 12, 15, 18, 19, 20, 25, 50, 75, 100, 125, 130, 135, 140, 145, 150, 160, 175 200, 225, 250, 275, 300, 350, 400, 450, 500, 550, 600, 650, 700, 750, 800, 850, 900, 950, or 1000 contiguous amino acids selected from SEQ ID NO:2 or any integer value between 10-1024 amino acids of SEQ ID NO: 2.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, Asp-Tyr-Lys-Asp-Asp-Asp-Asp-Lys (FLAG) tag (SEQ ID NO: 12), Met-Ala-Ser-Met-Thr-Gly-Gly-Gln-Gln-Met-Gly (T7) tag (SEQ ID NO: 13), influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and herpes simplex virus (HSV) BP16 protein fusions.

These fusions can be made, for example, by covalently linking two protein segments or by standard procedures in the art of molecular biology. Recombinant DNA methods can be used to prepare fusion proteins, for example, by making a DNA construct which comprises coding sequences selected from SEQ ID NO:1 in proper reading frame with nucleotides encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

These heterologous polypeptides may be of any length and may include one or more amino acids. In certain embodiments, Ipaf fusion proteins may be produced to facilitate expression or purification. For example, an Ipaf polypeptide may be fused to maltose binding protein or glutathione-S-transferase. In other embodiments, Ipaf fusion proteins may contain an epitope tag to facilitate identification or purification. One example of a tag is the FLAG epitope tag (Kodak). Ipaf variants may have certain amino acids which have been deleted, replaced or modified. Variants can also include post-translational modifications, and Ipaf or derivatives thereof can be glycosylated or unglycosylated.

In certain embodiments, the heterologous polypeptides are encoded by heterologous polynucleotides. A “heterologous” region of a DNA or RNA construct is an identifiable segment of DNA or RNA molecule within a larger nucleic acid that is not found in association with the larger molecule in nature. For instance, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The CARD domain of several apoptotic proteins contains a conserved leucine residue essential for dimerization (Duan and Dixit, Nature 385:86-89, 1997; Shaham et al., Genes Dev. 10:578-591, 1996). This leucine residue is also conserved in the CLAP proteins and is required for dimerization of the CLAP CARD domain. The NBD domain is designated for its capacity of binding nucleotides. The leucine-rich repeat (LRR) is a structural motif of about 20 to 29 amino acid residues in length associated with protein-protein interactions. X-ray structure determination of LRR motifs suggests that each LRR is composed of a β-sheet and an α-helix. The LRR domain is capable of interacting specifically with another LRR domain, thereby mediating protein-protein interactions.

C. Vectors, Host Cells and Means of Expressing and Producing Protein

The present invention encompasses vectors comprising regulatory elements linked to Ipaf nucleic acid sequences. Such vectors may be used, for example, in the propagation and maintenance of Ipaf nucleic acid molecules or the expression and production of Ipaf polypeptides and nucleic acid molecules. Vectors may include, but are not limited to, plasmids, episomes, baculovirus, retrovirus, lentivirus, adenovirus, and parvovirus including adeno-associated virus. A “vector” is a replicon in which another polynucleotide segment is attached, such as to bring about the replication and/or expression of the attached segment. The term includes expression vectors, cloning vectors, and the like.

Ipaf may be expressed in a variety of host organisms. In certain embodiments, Ipaf is produced in mammalian cells, such as CHO, COS-7, or 293 cells. Other suitable host organisms include bacterial species (e.g., E. coli and Bacillus), other eukaryotes such as yeast (e.g., Saccharomyces cerevisiae), plant cells and insect cells (e.g., Sf9). Vectors for these hosts are well known in the art.

A DNA sequence encoding Ipaf, or a fragment or variant thereof, is introduced into an expression vector appropriate for a host. The sequence is derived from an existing clone or synthesized. As described herein, a fragment of the coding region may be used. A preferred means of synthesis is amplification of the gene from cDNA, genomic DNA, or a recombinant clone using a set of primers that flank the coding region or the desired portion of the protein. Restriction sites are typically incorporated into the primer sequences and are chosen with regard to the cloning site of the vector. If necessary, translational initiation and termination codons can be engineered into the primer sequences. The sequence of Ipaf can be codon-optimized for expression in a particular host. For example, an Ipaf isolated from a human cell that is expressed in a fungal host, such as yeast, can be altered in nucleotide sequence to use codons preferred in yeast. Further, it may be beneficial to insert a traditional AUG initiation codon at the CUG initiation positions to maximize expression, or to place an optimized translation initiation site upstream of the CUG initiation codon. Accordingly, such codon-optimization may be accomplished by methods such as splice overlap extension, site-directed mutagenesis, automated synthesis, and the like.

The DNA sequence encoding the Ipaf may be fused to a heterologous polynucleotide sequence. A “heterologous” region of a DNA or RNA construct is an identifiable segment of DNA or RNA molecule within a larger nucleic acid that is not found in association with the larger molecule in nature. For instance, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

At minimum, the vector must contain a promoter sequence. As used herein, a “promoter” refers to a nucleotide sequence that contains elements that direct the transcription of a linked gene. At minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operably linked”. “Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequences. A coding sequence may be operably linked to control sequences that direct the transcription of the polynucleotide whereby said polynucleotide is expressed in a host cell.

Typical regulatory elements within vectors include a promoter sequence which contains elements that direct transcription of a linked gene and a transcription termination sequence. The promoter may be in the form of a promoter which is naturally associated with the gene of interest. Alternatively, the nucleic acid may be under control of a heterologous promoter not normally associated with the gene. For example, tissue specific promoter/enhancer elements may be used to direct expression of the transferred nucleic acid in repair cells. In certain instances, the promoter elements may drive constitutive or inducible expression of the nucleic acid of interest. Mammalian promoters may be used, as well as viral promoters capable of driving expression in mammalian cells. Examples of other regulatory elements which may be present include secretion signal sequences, origins of replication, selectable markers, recombinase sequences, enhancer elements, nuclear localization sequences (NLS) and matrix association regions (MARS).

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, Ipp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009) and the like.

The promoter controlling transcription of Ipaf may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to, the E. coli lacI repressor responsive to IPTG induction, the temperature sensitive λcl857 repressor, and the like. The E. coli lacI repressor is preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

Preferably, the vector is capable of replication in a host cell. Thus, when the host cell is a bacterium, the vector preferably contains a bacterial origin of replication. Preferred bacterial origins of replication include the f1-ori and col E1 origins of replication, especially the ori derived from pUC plasmids. In yeast, ARS or CEN sequences can be used to assure replication. A well-used system in mammalian cells is SV40 ori.

The vectors also preferably include at least one selectable marker that is functional in a host cell. A selectable marker gene includes any gene that confers a phenotype on the host cell that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance). Other selectable markers include genes that express products allowing for detection of transformed cells by other methods, such as fluorescence activated cell sorting. Examples of such markers include genes encoding β-galactosidase and green fluorescent protein.

The sequence of nucleotides encoding Ipaf may also include a secretion signal, whereby the resulting peptide is a precursor protein processed and secreted. The resulting processed protein may be recovered from the periplasmic space, the growth medium, phloem, etc. Secretion signals suitable for use are widely available and are well known in the art (von Heijne, J. Mol. Biol. 184:99-105, 1985). Prokaryotic and eukaryotic secretion signals that are functional in E. coli (or other host) may be employed. The presently preferred secretion signals include, but are not limited to, those encoded by the following E. coli genes: pelB (Lei et al., J. Bacteriol. 169:4379, 1987), phoA, ompA, ompt, ompF, ompC, beta-lactamase, and alkaline phosphatase.

One skilled in the art appreciates that there are a wide variety of suitable vectors for expression in bacterial cells which are readily obtainable. Vectors such as the pET series (Novagen, Madison, Wis.), the tac and trc series (Pharmacia, Uppsala, Sweden), pTTQ18 (Amersham International pic, England), pACYC 177, the pGEX series, and the like are suitable for expression of Ipaf. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see U.S. Pat. No. 4,745,051), for example. The choice of a bacterial host for the expression of Ipaf is dictated in part by the vector. Commercially available vectors are paired with suitable hosts.

A wide variety of suitable vectors for expression in eukaryotic cells are also available. Such vectors include pCMVLacI, pXT1 (Stratagene Cloning Systems, La Jolla, Calif.); pCDNA series, pREP series, and pEBVHis (Invitrogen, Carlsbad, Calif.), for example. In certain embodiments, Ipaf gene is cloned into a gene targeting vector, such as pMC1 neo, a pOG series vector (Stratagene Cloning Systems).

Ipaf may be isolated by standard methods, such as affinity chromatography, size exclusion chromatography, metal ion chromatography, ionic exchange chromatography, HPLC, and other known protein isolation methods. (See generally Ausubel et al., supra; Sambrook et al., supra). An isolated purified protein usually gives a single band on SDS-PAGE when stained with Coomassie blue.

Ipaf, as discussed earlier, may be expressed as a fusion protein to aid in purification. Such fusions may be, for example, glutathione-S-transferase fusions, Hex-His fusions, or the like such that the fusion construct may be easily isolated. With regard to Hexa-His fusions, such fusions can be isolated by metal-containing chromatography, such as nickel-coupled beads. Briefly, a sequence encoding His₆ is linked to a DNA sequence encoding Ipaf. Although the His₆ sequence can be positioned anywhere in the molecule, preferably it is linked at the 3′ end immediately preceding the termination codon. The fusion may be constructed by any of a variety of methods. A convenient method is amplification of the Ipaf gene using a downstream primer that contains the codons for His₆.

Purified Ipaf protein may be used as a therapeutic or in various assays to screen for modulators (i.e., inhibitors or enhancers) of apoptosis. These assays may be performed in vitro or in vivo and utilize any of the methods described herein or that are known in the art. The protein may also be crystallized and subjected to X-ray analysis to determine its 3-dimensional structure. Ipaf polypeptides may also be used as immunogens for raising antibodies. Further, antibodies to Ipaf can be used diagnostically to determine the presence or absence of various Ipaf isoforms in a sample.

Recombinant Ipaf may be produced by expressing DNA sequences provided in the invention. Using methods known in the art, an Ipaf expression vector may be constructed, transformed into a suitable host cell, and conditions suitable for expression of Ipaf by the host cell established. One skilled in the art will appreciate that there are a wide variety of suitable vectors for expression in bacterial cells (e.g. pET series (Novagen, Madison, Wis.)), insect cells (e.g. pBlueBac (Invitrogen, Carlsbad, Calif.)), and eukaryotic cells (e.g. pCDNA and pEBVHis (Invitrogen, Carlsbad, Calif.)). In certain embodiments, the Ipaf nucleic acid molecule may be cloned into a gene targeting vector such as pMC1neo (Stratagene, La Jolla, Calif.). Synthetic chemistry methods, such as solid phase peptide synthesis can also be used to synthesize proteins, fusion proteins, or polypeptides of the invention.

The resulting expressed protein can be purified from the culture medium or from extracts of the cultured cells. Methods of protein purification such as affinity chromatography, ionic exchange chromatography, HPLC, size exclusion chromatography, ammonium sulfate crystallization, electrofocusing, or preparative gel electrophoresis are well known an widely used in the art (see generally Ausubel et al., supra; Sambrook et al., supra). An isolated purified protein is generally evidenced as a single band on an SDS-PAGE gel stained with Coomassie blue.

D. Anti-Ipaf Antibodies

Antibodies that have specificity for an Ipaf polypeptide or functional fragment thereof are provided by the present invention. Antibodies of the invention can be used, for example, to detect Ipaf polypeptides. The antibodies can be used for isolation of Ipaf polypeptides and in the identification of molecules that interact with Ipaf polypeptides. The antibodies may also be used to inhibit or enhance the biological activity of Ipaf polypeptides and for diagnostic or therapeutic purposes.

Within the context of the current invention, antibodies are understood to include monoclonal antibodies, polyclonal antibodies, anti-idiotypic antibodies, antibody fragments (e.g., Fab and F(ab′)₂, Fᵥ variable regions, or complementarity determining regions), single chain antibodies and humanized antibodies. Antibodies are generally accepted as specific to Ipaf proteins if they bind with a K_{d} of greater than or equal to 10⁻⁷M, and preferably 10⁻⁸M. The affinity of an antibody can be readily determined by one of ordinary skill in the art (see Skatchard, Ann. N.Y. Acad. Sci. 51:660-672, 1949).

Antibodies may be produced by any of a variety of methods available to one of ordinary skill in the art. Detailed methods for generating antibodies are provided in Antibodies: A Laboratory Manual, Harlow and Lane (eds.), Cold Spring Harbor Laboratories, 1988, which is incorporated by reference.

A polyclonal antibody may be readily generated in a variety of animals such as rabbits, mice and rats. Generally, an animal is immunized with an Ipaf polypeptide or one or more peptides comprising Ipaf amino acid sequences which may be conjugated to a carrier protein. Routes of administration include intraperitoneal, intramuscular, intraocular, or subcutaneous injections, usually in an adjuvant (e.g., Freund's complete or incomplete adjuvant).

Monoclonal antibodies may be readily generated from hybridoma cell lines using conventional techniques (see Antibodies: A Laboratory Manual, Harlow and Lane (eds.), Cold Spring Harbor Laboratories, 1988). Various immortalization techniques such as those mediated by Epstein-Barr virus or fusion to produce a hybridoma may be used. In a preferred embodiment, immortalization occurs by fusion with a myeloma cell line (e.g., NS-1 (ATCC No. TIB 18) and P3×63-Ag 8.653 (ATCC No. CRL 1580)) to create a hybridoma that secretes a monoclonal antibody.

Antibody fragments, such as Fab and Fv fragments, may be constructed, for example, by conventional enzymatic digestion or recombinant DNA techniques to yield isolated variable regions of the antibody. Within one embodiment, the genes which encode the variable region from a hybridoma producing a monoclonal antibody of interest are amplified using nucleotide primers corresponding to the variable region. Amplification products are subcloned into plasmid vectors and propagated and purified using bacteria, yeast, plant or mammalian-based expression systems. Techniques may be used to change a murine antibody to a human antibody, known familiarly as a “humanized” antibody, without altering the binding specificity of-the antibody.

Antibodies may be assayed for immunoreactivity against Ipaf by any of a number of methods, including western blot, enzyme-linked immuno-sorbent assays (ELISA), countercurrent immuno-electrophoresis, radioimmunoassays, dot blot assays, sandwich assays, inhibition or competition assays, or immunoprecipitation (see U.S. Pat. Nos. 4,376,110 and 4,486,530; see also Antibodies: A Laboratory Manual, Harlow and Lane (eds.), Cold Spring Harbor Laboratory Press, 1988). Techniques for purifying antibodies are those available in the art. In certain embodiments, antibodies are purified by passing the antibodies over an affinity column to which amino acid sequences of the present invention are bound. Bound antibody is then eluted. Other purification techniques include, but are not limited to HPLC or RP-HPLC, or purification on protein A or protein G columns.

E. Methods of Using Ipaf Nucleic Acids AND Polypeptides

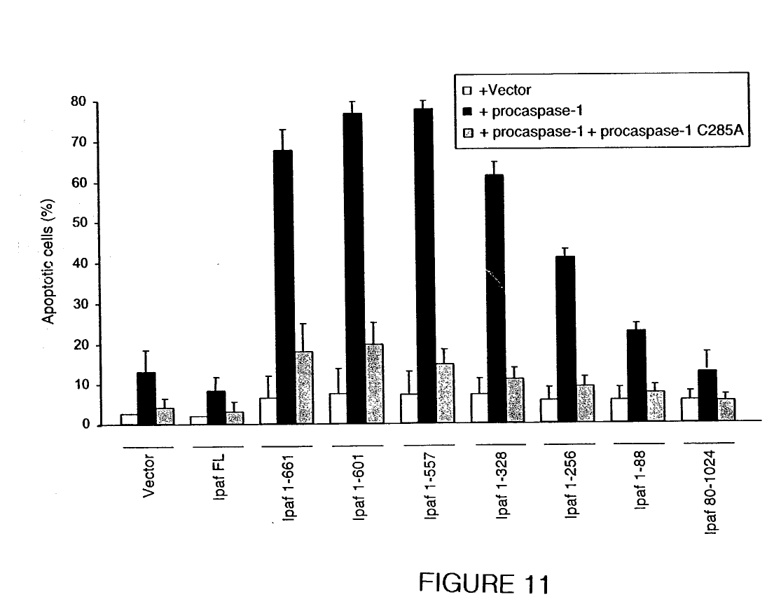

Two readily observable functions of Ipaf polypeptides or functional fragments thereof are induction of apoptosis by activating the caspase-1 pathway and subsequent modulation of inflammation through IL-1β (FIG. 11). Thus, the compositions described herein, including Ipaf nucleic acids, polypeptides and antibodies, can be used for a variety of assay, therapeutic, and diagnostic purposes.

1. Identification of Inhibitors and Enhancers of Ipaf Mediated Apoptotic Activity

Candidate inhibitors and enhancers may be isolated or procured from a variety of sources, such as bacteria, fungi, plants, parasites, libraries of chemicals, peptides or peptide derivatives, and the like. Inhibitors and enhancers may be also be rationally designed, based on the protein structures determined from X-ray crystallography. In certain preferred embodiments, the inhibitor or enhancer targets the CARD/CARD interaction responsible for Ipaf dimerization.

Inhibitors of apoptotic activity may be desirable in certain circumstances, such as for the treatment of a patient suffering from a disease resulting from increased apoptosis. Without wishing to be bound to a particular theory or held to a particular mechanism, an inhibitor may act by preventing Ipaf induced processing of caspase or by preventing enzymatic activity, by inhibiting Ipaf/Ipaf complex formation or Ipaf/procaspase-1 complex formation, or by other mechanisms. The inhibitor may act directly or indirectly. In preferred embodiments, inhibitors interfere in the Ipaf mediated processing of the caspase protein, the Ipaf/Ipaf oligomerization and/or the integrity of the complex, or may modulate Ipaf phosphorylation. In certain embodiments, the inhibitors are small molecules. In one embodiment, the inhibitors prevent apoptosis. Inhibitors should have a minimum of side effects and are preferably non-toxic.

In addition, enhancers of apoptotic activity are desirable in certain circumstances. At times, increasing apoptosis will have a therapeutic effect. For example, tumors or cells that mediate autoimmune diseases are appropriate cells for destruction. Enhancers may increase the rate or efficiency of caspase processing, increase transcription or translation, decrease proteolysis, or act through other mechanisms, for example. As will be apparent to those skilled in the art, many of the guidelines presented above apply to the design of enhancers as well. Within the context of the present invention, Ipaf itself can act as an enhancer. Further, other compounds which facilitate Ipaf oligomerization are reasonably expected to enhance apoptosis. For example, Ipaf proteins or fragments such as the CARD or NBT domains fused to Fc domains, FKBP (FK506 binding protein), or the like, can form oligomers which lead to caspase activation.

Screening assays for inhibitors and enhancers will vary according to the type of inhibitor or enhancer and the nature of the activity that is being affected. Assays may be performed in vitro or in vivo. In general, assays are designed to evaluate apoptotic pathway activation (e.g., caspase protein processing, caspase enzymatic activity, cell morphology changes, DNA laddering, and the like). In any of the assays, a statistically significant increase or decrease compared to a proper control is indicative of enhancement or inhibition. In one embodiment, the caspase utilized for the assays is procaspase-1/caspase-1.

One in vitro assay can be performed by examining the effect of a candidate compound on the activation of an effector caspase (e.g., caspases 3-7). The processing of procaspase-1 into two subunits can be assayed or alternatively caspase-1 enzymatic activity can be monitored by adding procaspase-3, procaspase-6, or other effector caspases and monitoring the activation of these caspases either directly via substrate turnover (e.g., Acetyl DEVD-aminomethyl coumarin (amc), lamin, PRPP, PARP, and the like) or via subunit formation, for example.

Further, inhibition or enhancement of Ipaf oligomerization with another Ipaf molecule or procaspase-1 or a binding portion thereof can be monitored in the presence or absence of candidate inhibitors or enhancers. Such constructs can be readily tested for oligomerization by known methods of detecting protein-protein binding. For example, one can utilize sedimentation analysis, electrophoretic gel shift analysis, radiolabeled polypeptide binding studies, and the like. Further, to facilitate detection, typically the protein of interest may be in vitro translated and labeled during translation. This composition is incubated with an Ipaf polypeptide in the presence or absence of a candidate inhibitor or enhancer. Processing of procaspase-1 into two subunits can be monitored as can processing/activation of a coincubated effector pro-caspase. A processible fragment of procaspase-1 may also be utilized to monitor the processing of procaspase-1, wherein the processible fragments refer to any fragment of procaspase-1 that can be processed by the Ipaf. Caspase processing is routinely monitored either by gel electrophoresis or indirectly by monitoring caspase substrate turnover. The two subunits and caspase substrate turnover may be readily detected by autoradiography after gel electrophoresis. One skilled in the art will recognize that other methods of labeling and detection may be used alternatively.

Moreover, any known enzymatic analysis can be used to follow the inhibitory or enhancing ability of a candidate compound with regard to the ability of Ipaf or fragments thereof to produce enzymatically active caspases. For example, one could express an Ipaf construct of interest in a cell line, be it bacterial, insect, mammalian or other, and purify the resulting polypeptide. The purified Ipaf polypeptide can then be used in a variety of assays to follow its ability to facilitate processing or catalytic ability of effector caspases, or apoptotic activity. Such methods of expressing and purifying recombinant proteins are known in the art and examples can be found in Sambrook et al., Molecular Cloning: A Laboratory Manual, Cold Spring Harbor Press, 1989, as well as in a number of other sources.

In vivo assays are typically performed in cells transfected either transiently or stably with an expression vector containing the Ipaf nucleic acid molecule such as those described herein. These cells are used to measure caspase processing, caspase substrate turnover, or apoptosis in the presence or absence of a candidate compound. When assaying apoptosis, a variety of cell analyses may be used including, for example, dye staining and microscopy to examine nucleic acid fragmentation, porosity of the cells, and membrane blebbing.

A variety of methodologies exist that can be used to investigate the effect of a candidate compound. Such methodologies are those commonly used to analyze enzymatic reactions and include, for example, SDS-PAGE, spectroscopy, HPLC analysis, autoradiography, chemiluminescence, chromogenic reactions, and immunochemistry (e.g., blotting, precipitating, etc.).

Inhibitors and enhancers may also be used in the context of this invention to exert control over the cell death process or cytokine activation (e.g., IL-1, which is activated by caspase-1). Thus, these inhibitors and enhancers will have utility in the treatment of diseases characterized by either excessive or insufficient levels of apoptosis and/or inflammation. Inhibitors of caspase activation may be used to treat the major neurodegenerative diseases, including stroke, Parkinson's Disease, Alzheimer's Disease, and ALS, for example. As well, caspase activation inhibitors may be used to inhibit apoptosis in the heart following myocardial infarction, in the kidney following acute ischemia, and in diseases of the liver. Enhancers of caspase activation may be used in contexts where apoptosis or cytokine activation are desired. For example, inducing or increasing apoptosis in cancer cells or aberrantly proliferating cells may be effected by delivery of a caspase enhancer. In this regard, Ipaf polypeptides can function as enhancers of apoptosis when introduced into a cell.

The inhibitors and enhancers may be further coupled with a targeting moiety that binds a cell surface receptor specific to the cells. Administration of inhibitors or enhancers will generally follow established protocols. The compositions of the present invention may be administered either alone, or as a composition. Briefly, compositions of the present invention may comprise one or more of the inhibitors or enhancers as described herein, in combination with one or more biologically, pharmaceutically, or physiologically acceptable carriers, diluents or excipients. Such compositions may comprise buffers such as neutral buffered saline, phosphate buffered saline, and the like, carbohydrates such as glucose, mannose, sucrose or dextrans, mannitol, proteins, polypeptides or amino acids such as glycine, antioxidants, chelating agents such as EDTA or glutathione, adjuvants (e.g., aluminum hydroxide) and preservatives. In addition, compositions of the present invention may also contain one or more additional active ingredients.

Compositions of the present invention may be formulated for the manner of administration indicated, including for example, for oral, nasal, venous, intracranial, intraperitoneal, subcutaneous, or intramuscular administration. Within other embodiments of the invention, the compositions described herein may be administered as part of a sustained release implant. Within yet other embodiments, compositions of the present invention may be formulized as a lyophilizate, utilizing appropriate excipients which provide stability as a lyophilizate, and subsequent to rehydration. One skilled in the art may further formulate the enhancers or inhibitors of this invention in an appropriate manner, and in accordance with accepted-practices, such as those disclosed in Remington's Sciences, Gennaro, Ed., Mack Publishing Co., Easton, Pa. 1990. Appropriate dosage amounts balancing toxicity and efficacy will be determined during clinical testing, but will typically include a dosage ranging from 0.1 to 100 mg/kg of the subject. When using gene therapeutics, such dosages will depend on the vector utilized and will be determined during clinical trials.

2. Enhancement or Inhibition of Apoptosis

Compositions comprising an Ipaf polynucleotide or polypeptide, or a fragment thereof, are provided by the invention. Such compositions may be used to inhibit or promote apoptosis or procaspase-1 processing, for example. In certain embodiments, compositions comprising the CARD domain, the NBD domain or the LRR domain of an Ipaf protein are provided.

In certain other embodiments, compositions of the invention comprise antibodies that selectively bind to an Ipaf polypeptide. These antibodies include, but are not limited to, polyclonal, monoclonal, single chain or humanized antibodies or antibody fragments. They may be specific for human Ipaf, or they may recognize Ipaf polypeptides of other species, also. These compositions may comprise, for example, polyclonal antibodies which recognize one or more epitopes within the Ipaf protein. Alternatively, they can comprise monoclonal antibodies which recognize specific epitopes within an Ipaf polypeptide. The antibodies of the composition may recognize native Ipaf proteins and/or denatured Ipaf polypeptides. These antibodies may be produced according to methods well known in the art, as described above.

Examples of polynucleotide compositions include mammalian expression vectors, sense RNAs, ribozymes, antisense RNA expressing vectors, shRNAs, siRNAs, dsRNAs, and antisense RNAs. Expression vectors and sense RNA molecules are designed to express Ipaf polypeptides, while ribozymes and antisense RNA constructs are designed to reduce the levels of Ipaf polypeptides expressed, as described above. Polynucleotide compositions may comprise the entire Ipaf open reading frame or cDNA, or may contain only a fragment of the full length Ipaf nucleic acid sequence or variant thereof.

The compositions may also contain a physiologically acceptable carrier. The term “physiologically acceptable carrier” refers to a carrier for administration of a therapeutic agent such as antibodies, polypeptides or nucleic acids. Suitable carriers and physiologically acceptable salts are well known to those of ordinary skill in the art. A thorough discussion of acceptable carriers is available in Remington's Sciences, Mack Publishing Co., NJ, 1991).

The term “effective amount” refers to an amount of an agent effective in treating, ameliorating, or preventing a specific disease or condition, or which produces a detectable therapeutic or preventative effect. The effect can be detected by, for example, chemical markers or antigen levels. Detectable effects also include reduction in disease-related physical symptoms. The effective amount for a given disease and patient can be determined by routine experimentation and is within the judgment of the clinician. The precise effective amount will vary depending on factors including, but not limited to, the subject's size and health, the nature and extent of the condition or disease, and the particular therapeutic compound. Exemplary doses will likely be from about 0.001 mg/kg to 50 mg/kg or 0.05 mg/kg to about 10 mg/kg of the polynucleotide, polypeptide or antibody compositions in the individual to which it is administered. Compositions containing antisense Ipaf polynucleotides are generally administered in a range of about 100 ng to about 200 mg of polynucleotides for local administration in a gene therapy protocol. The effective dosages for compositions containing protein, polypeptide or antibody are in the range of about 5 μg to about 50 μg/kg of patient body weight, about 50 μg to about 5 mg/kg, about 100 μg to about 500 μg/kg of patient body weight, and about 200 to about 250 μg/kg. In all cases, routine experimentation in clinical trials will determine specific ranges for optimal therapeutic effects.

Polynucleotide and polypeptide compositions of the invention can be (1) administered directly to the subject; (2) delivered ex vivo to cells derived from the subject; or (3) delivered in vitro for expression of recombinant proteins. Direct delivery will generally be accomplished by injection. Alternatively, compositions can also be delivered via oral or pulmonary administration, suppositories, transdermally, or by gene guns, for example. Dosage treatment may be a single dose or multiple doses.

Methods of ex vivo delivery and reimplantation of transformed cells into a subject are known in the art. Generally, delivery of nucleic acids for both ex vivo and in vitro applications can be accomplished by, for example, dextran-mediated transfection, calcium phosphate precipitation transfection, viral infection, polybrene mediated transfection, protoplast fusion, electroporation, encapsulation of polynucleotides in liposomes, and direct microinjection of the DNA into nuclei, all well known in the art.

Gene therapy vectors comprising Ipaf nucleic acid sequences, or complements or variants thereof, are within the scope of the invention. These vectors may be used to regulate Ipaf mRNA and polypeptide expression in target cells. In some instances, it may be advantageous to increase the amount of Ipaf nucleic acids or polypeptides which are expressed. In other cases, gene therapy vectors may be used to decrease functional Ipaf polypeptide levels. Gene therapy vectors may comprise any Ipaf nucleic acid of the current invention, including fragments, variants, antisense, ribozymes, and mutants. Expression of Ipaf nucleic acids may be controlled by endogenous mammalian or heterologous promoters and may be either constitutive or regulated. Nucleic acids used according to the invention may be stably integrated into the genome of the cell or may be maintained in the cell as separate episomal segments of DNA.

Ipaf nucleic acid molecules may be delivered by any method of gene delivery available in the art. Gene delivery vehicle may be of viral or non-viral origin (see generally Jolly, Cancer Gene Therapy 1:51-64, 1994; Kimura, Human Gene Therapy 5:845-852, 1994; Connelly, Human Gene Therapy 1:185-193, 1995; and Kaplitt, Nature Genetics 6:148-153, 1994). The present invention can employ recombinant retroviruses which are constructed to carry or express an Ipaf nucleic acid molecule. Methods of producing recombinant retroviral virions suitable for gene therapy have been extensively described (see, e.g, Mann et al., Cell 33:153-159, 1983; Nikolas and Rubenstein, Vectors: A survey of molecular cloning vectors and their uses, Rodriquez and Denhardt (eds.), Stoneham:Butterworth, 494-513, 1988).

The present invention also employs viruses such as alphavirus-based vectors, adenovirus, retrovirus, and parvovirus that can function as gene delivery vehicles. Examples of vectors utilized by the invention include intact adenovirus, replication-defective adenovirus vectors requiring a helper plasmid or virus, and adenovirus vectors with their native tropism modified or ablated such as adenoviral vectors containing a targeting ligand. Other examples include adeno-associated virus based vectors and lentivirus vectors.

Packaging cell lines suitable for use with the above-described viral and retroviral vector constructs may be readily prepared and used to create producer cell lines (also termed vector cell lines) for the production of recombinant vector particles.

Examples of non-viral methods of gene delivery vehicles and methods which may be employed according to the invention include liposomes (see, e.g., Wang et al., PNAS 84:7851-7855, 1987), polycationic condensed DNA (see, e.g., Curiel, Hum. Gene Ther. 3:147-154, 1992); ligand linked DNA (see, e.g., Wu, J. Biol. Chem. 264:16985-16987, 1989); deposition of photopolymerized hydrogel materials; hand-held gene transfer particle guns, as described in U.S. Pat. No. 5,149,655; ionizing radiation as described in U.S. Pat. No. 5,206,152 and WO 92/11033; and nucleic charge neutralization or fusion with cell membranes. Additional approaches are described in Philip, Mol. Cell Biol. 14:2411-2418, 1994 and in Woffendin et al., Proc. Natl. Acad. Sci. USA 91:1581-1585, 1994. Conjugates comprising a receptor-binding internalized ligand capable of delivering nucleic acids may also be used according to the present invention. Conjugate-based preparations and methods of use thereof are described in U.S. Pat. No. 6,037,329 which is hereby incorporated by reference in its entirety. Other non-viral delivery methods include, but are not limited to, mechanical delivery systems such as the approach described in Woffendin et al., Proc. Natl. Acad. Sci. USA 91: 1581-1585, 1994 and naked DNA protocols. Exemplary naked DNA introduction methods are described in WO 90/11092 and U.S. Pat. No. 5,580,859.

In other embodiments, methods of the invention utilize bacteriophage delivery systems capable of transfecting eukaryotic cells. Bacteriophage-mediated gene transfer systems are described in U.S. Pat. No. 6,054,312, which is incorporated in its entirety. Phage delivery vehicles may express a targeting ligand on their surface, which facilitates receptor-mediated gene delivery.

The compositions of the invention can be used to treat a mammal with any of a variety of diseases including, for example, cancer, tumor progression, hyperproliferative cell growth, immune disorder, autoimmune disease, neurodegenerative disease, ischemic injury, hepatic injury, or accompanying biological and physical manifestations. Methods of treatment with polynucleotides including, for example, antisense constructs, ribozyme constructs, and expression vectors comprising Ipaf polynucleotide sequences or complements or variants thereof, are in the scope of the invention. Similarly, methods of treatment with polypeptides including, for example, Ipaf polypeptides or fragments or variants thereof, or Ipaf antibodies, are in the scope of the invention. In addition, therapeutic compositions and methods of treatment using small molecule agonists or antagonists, dominant negative Ipaf polypeptides, or heterologous polypeptides which bind Ipaf polypeptides are included within the scope of the current invention.

3. Detection of Apoptosis and Apoptosis-Related Disease

Ipaf polynucleotides and polypeptides are implicated in apoptosis. Thus, Ipaf polypeptides are likely to play a role is cellular processes regulated by apoptosis. One example of such a process is cell proliferation, since apoptosis plays an important role in maintaining homeostasis of cell growth. Therefore, Ipaf molecules are likely to play a role in diseases affecting cell proliferation such as cancer, tumor progression or metastasis, and hyperproliferative cell growth. Deregulated Ipaf expression or expression of an Ipaf mutant or variant can result in too little or too much apoptosis, leading to either hyperproliferation or inappropriate cell death. Other cell process regulated by apoptosis include inflammation and immune responses. Generally, these processes involve infiltration of immune cells and subsequent clearing of such cells via mechanisms including apoptosis. Additionally, autoreactive T lymphocytes are deleted by apoptosis. Any disruption of normal apoptosis can lead to the production of autoreactive T cells and to the maintenance of inflammatory and immune cells normally cleared via apoptosis, thus contributing to a variety of disease conditions including autoimmune diseases such as rheumatoid arthritis and lupus erythematosus, and other immune and inflammatory disorders such as transplant rejection, rheumatic disorders including dermatomyositis, AIDS, acute pancreatitis, and immune-mediated bowel disorders. Similarly, aberrant apoptosis has been implicated in neurological diseases. All diseases associated with apoptosis are candidates for therapeutic treatment with the compositions and methods of the current invention.

Ipaf polynucleotides, complements thereof, and antibodies which bind Ipaf polypeptides can be used as markers to diagnose and determine the prognosis of cancer, tumor progression, hyperproliferative cell growth, immune disorders, autoimmune diseases, neurodegenerative diseases, ischemic injury, hepatic injury, or accompanying biological and physical manifestations. Levels of Ipaf polynucleotides or polypeptides in a sample are compared to the levels in a normal control sample. The normal sample can include a pool of cells from a particular tissue or tissues and/or cells from throughout the body or may include cells of the same or a different type from another individual, for example. Immunoassays or nucleic acid assays can be used for such measurements. Any observed difference between the sample and the normal control can indicate the occurrence of disease or disorder.

Nucleic acid assays utilize subgenomic polynucleotides capable of hybridizing under stringent conditions to Ipaf polynucleotides or complements thereof. Polynucleotide probes comprising at least 10 contiguous nucleotides selected from the nucleotide sequence of Ipaf are labeled, for example, with radioactive, fluorescent, biotinylated, or chemiluminescent label, and detected by well known methods appropriate for the particular label selected. Subgenomic polynucleotides are preferably intron-free. Polynucleotides corresponding to Ipaf can be propogated in suitable vectors and hosts. Ipaf polynucleotides can be isolated from DNA vectors by standard techniques, such as restriction enzyme digestion and gel electrophoresis or chromatography. The polynucleotides can also be produced using the polymerase chain reaction (PCR) according to methods available in the art.

The Ipaf polynucleotides can be used to compare related genes in normal control tissue and suspected diseased tissue by any means known in the art. For example, the Ipaf from a suspected diseased tissue can be sequenced and compared with the Ipaf sequence in the normal tissue. Ipaf genes, or portions thereof, can be amplified, for example, using nucleotide primers based on the disclosed human Ipaf sequence, using the polymerase chain reaction (PCR). The amplified nucleic acid molecules can be sequenced according to any method known in the art such as, for example, the dideoxy sequencing method. Nucleotide probes and nucleotides incorporated during sequencing are labeled by a variety of methods, such as radiolabeling, biotinylation, or labeling with fluorescent or chemiluminescent tags, and detected by standard methods known in the art. A difference in the nucleotide sequence of the Ipaf from the suspected diseased tissue compared to the Ipaf from the normal control sample suggests a role for an encoded Ipaf polypeptide in the disease and provides a lead for preparing a therapeutic agent.

Alternatively, Ipaf mRNA levels in normal and suspected diseased tissues are compared. For example, polyA+ RNA is isolated from the two tissues as is known in the art. One of skill in the art can then readily determine differences in the size or amount of Ipaf-related mRNA transcripts between the two tissues by Northern blot analysis, primer extension, S1 nuclease protection assays, reverse transcription-polymerase chain reaction (RT-PCR), or in situ hybridization using polynucleotide probes corresponding to Ipaf or complements thereof. Increased or decreased expression of an Ipaf-related mRNA in a tissue sample suspected of being diseased, compared to the expression of the same Ipaf-related mRNA in a normal tissue, suggests that the expressed protein has a role in the disease and also provides a lead for preparing a therapeutic agent.

Ipaf gene expression can also be examined using polynucleotide arrays. Polynucleotide arrays provide a high throughput technique that can assay large numbers of polynucleotide sequences in a sample. Techniques for constructing arrays and methods of using these arrays are described in U.S. Pat. Nos. 5,593,839, 5,578,832, 5,599,695, 5,556,752, and 5,631,734, which are incorporated by reference.

Antibodies which bind Ipaf polypeptides can also be used for diagnostic purposes. These antibodies may be polyclonal, monoclonal, single chain antibodies, or humanized antibodies, for example, as described above.

Any method known in the art can be used to compare Ipaf proteins from normal control samples and suspected diseased samples. The size of the proteins in the two samples can be compared, for example, using antibodies against Ipaf polypeptides to detect Ipaf polypeptides by western blot. Alterations in the size of the Ipaf protein in a tissue suspected of being diseased compared with the level in a normal control sample indicate the protein is abnormal, possibly due to truncation, deletion, or altered post-translational modification. Size alterations are indicative that Ipaf has a role in the disease. Other changes, such as protein expression levels and subcellular localization can also be detected immunologically, for example, by using antibodies directed against polypeptides encoded by Ipaf for western blot or immunofluorescence. A higher or lower level of Ipaf protein in tissues suspected of being disease compared with the level in a normal control sample is indicative that Ipaf has a role in the disease. Similarly, changes in the subcellular localization of Ipaf protein also indicate Ipaf has a role in the disease.

Generally, changes in Ipaf polynucleotide sequence or expression levels or changes in Ipaf polypeptide size, expression levels, or subcellular localization that are associated with diseased tissue as compared to a normal control sample indicate that Ipaf plays a role in the etiology of the disease. Epidemiological studies can establish that alterations in Ipaf polynucleotides or polypeptides are associated with a particular disease. The identification of these alterations can be an indicator of the presence, prognosis or severity of a disease.

4. Diagnostic Kits

Diagnostic kits using materials and/or methods of the current invention are provided. Reagents specific for Ipaf polynucleotides and polypeptides, such as antibodies and polynucleotide probes, can be supplied in a kit for detecting the presence of an Ipaf expression product in a biological sample. The kit can also contain buffers or labeling components, detection reagents, instructions for using reagents to detect and quantify expression products in biological samples and control normal samples. Normal biological samples may be provided in any form suitable for the particular method of detection utilized by the kit. For example, normal biological samples can be polynucleotides, polypeptides, cellular extracts or tissue sections.

Components of the kit may be provided in a dried or lyophilized form or in one or more liquid solutions. When the components are provided in liquid solution, the liquid solution is preferably a sterile, aqueous solution. When components are provided in a dried form, the dried form may be capable of reconstitution upon addition of a suitable solvent.

Kits will generally be packaged in an outer container suitable for commercial sale and distribution. The container may be a box, or it may be an inhalant, syringe, pipette, dropper, or any other such apparatus, from which the formulation may be applied to a sample, or even applied to or mixed with other components of the kit.

Kits of the invention may also comprise or be packaged with instructions for use and instruments for assisting in use. Examples of instruments include syringes, inhalants, pipettes, vials, forceps, measuring spoons, eye droppers or any other medically approved delivery device.

From the foregoing it will be appreciated that, although specific embodiments of the invention have been described herein for purposes of illustration, various modifications may be made without deviating from the spirit and scope of the invention. In addition, all references including patents, patent applications, and journal articles are incorporated herein in their entirety. Accordingly, the invention is not limited except as by the appended claims.

### EXAMPLES

### Example 1

### Isolation of the Ipaf cDNA

In an attempt to isolate genes having sequence similarity to the CARD domain of caspase-1, a nucleotide sequence encoding a CARD containing peptide (GenBank™ accession number AL 121653) with homology to caspase-1 was found in the NCBI public genomic database using the TBLASTN program. Based on the predicted cDNA sequence of this gene, the complete cDNA of this new CARD-containing protein was isolated from a human peripheral blood leukocytes (PBL) cDNA library by a polymerase chain reaction (PCR) with two sets of primers corresponding to overlapping sequences of the coding region of the gene. The cDNA sequence of the gene was verified by nucleotide sequencing.

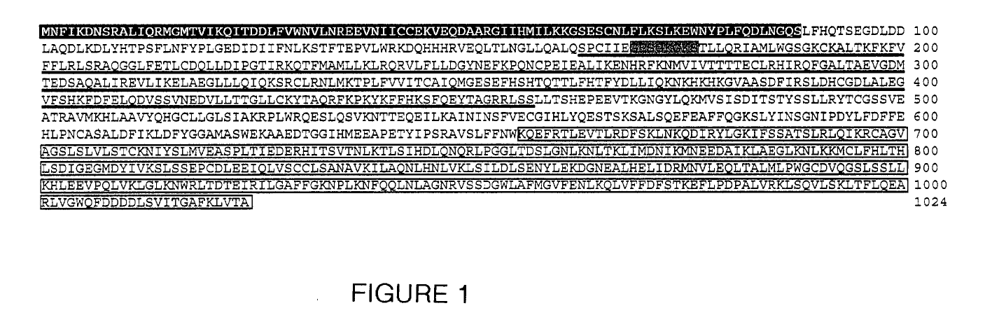

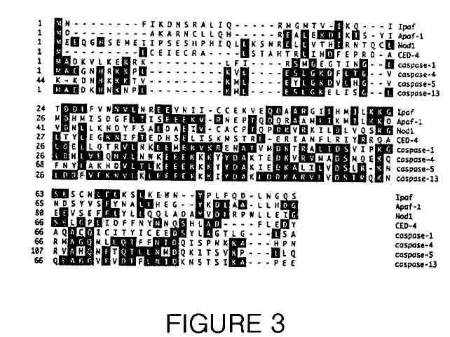

The nucleic acid and deduced amino acid sequences are presented in FIG. 1 as SEQ ID NO:1 and SEQ ID NO:2 respectively. Because of its activity as disclosed herein, this protein is termed as Ipaf for ICE-protease activating factor. Ipaf encodes a protein of 1024 residues with a predicted molecular mass of 116.1 kDa (See FIG. 1). A BLAST search of protein databases indicated that Ipaf is a novel protein composed of three putative functional domains (See, FIG. 2), including a NH₂-terminal CARD domain (residues 1-88), a putative nucleotide binding (NBD) domain (residues 163-457) and a C-terminal region (LRR) domain (residues 656-1024) containing multiple leucine-rich repeats. The CARD domain of Ipaf is mostly related to the CARD domains of caspase-1, and other caspase-1 related proteins (See, FIG. 3). The CARD/NBD/LRR domain structure of Ipaf is similar to that of CED-4/Apaf-1/Nod1, thus establishing Ipaf as a new member of the CED4/Apaf-1/Nod1 family of proteins.

### Example 2

### Differential Expression of Ipaf mRNA

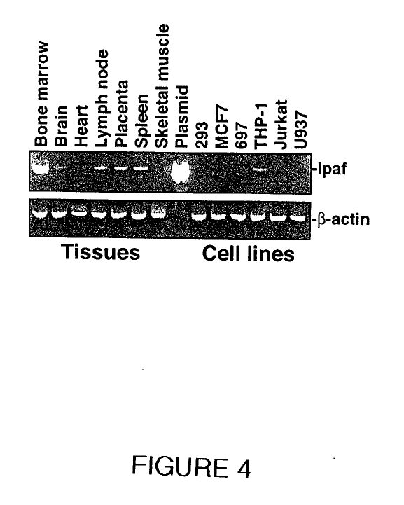

Total RNAs from different human tissues and cell lines were amplified by RT-PCR with primers specific for Ipaf or β-actin. A plasmid containing Ipaf was used as a control for the PCR reactions. PCR products were then analyzed on agarose gels and stained with ethidium bromides. FIG. 4 shows some representative results. RT-PCR analysis of the expression of the mRNA of Ipaf in multiple human tissues and cell lines revealed that Ipaf is highly expressed in bone marrow and to a lesser extent in lymph node, placenta and spleen. Ipaf mRNA was also detected in the brain. Among the different cell lines tested, Ipaf mRNA was found only THP-1, a monocytic cell line.

### Example 3

### Expression Vectors

Constructs encoding full length Ipaf or truncated mutants were generated by PCR using modified complementary PCR adapter-primers. Flag- and T7-epitope tagging was done by cloning the PCR generated cDNAs of the respective genes in-frame into pFLAG CMV-2 (IBI-Kodak) and pcDNA3-T7 (Invitrogen) vectors, respectively. Plasmids encoding GFP fusions were constructed using pEGFP-N1 (CLONTECH). The Ipaf-CARD-GST construct was made by cloning residues 1-88 of Ipaf with C-terminal GST in pET-28a (Novagen). Procaspase-1 and RICK constructs have been described previously (Inohara et al., J. Biol. chem. 273:12296-12300, 1998; Alnemri et al., J. Biol. Chem. 270:4312-4317, 1995).

### Example 4

### Cell Culture

Cells were cultivated either in Dulbecco's Modified Eagle Medium (DMEM) (MCF7 cells) or DMEM/F12 (293T cells) (Gibco), supplemented with 10% fetal bovine serum, 200 μg.ml⁻¹ penicillin and 100 μg.ml⁻¹ streptomycin sulfate.

### Example 5

### Confocal Microscopy

MCF7 cells were grown on coverslips and then transfected with constructs encoding GFP-tagged Ipaf. 24 hours following transfection, the cells were fixed with 4% paraformaldehyde in PBS for 30 min. The coverslips were mounted on a glass slide and the GFP fluorescence was detected by confocal microscopy using excitation wavelength of 488 nm and detection wavelength of 522 nm. Images were Kalman-averaged.

### Example 6

### Transfection, Immunoprecipitation, Immunoblot and GST Pull Down

293T cells (5×10⁶ cells) in 100-mm dishes were transiently transfected with expression plasmids using the LipofectAMINE™ (Life Technologies, Inc.) method as per the manufacturer's instructions. 24 hours following transfection, cells were lysed in 50 mM Tris/HCl pH 7.6, 150 mM NaCl containing 0.5% NP-40, 10 μg.ml⁻¹ leupeptin and aprotinin and 0.1 mM PMSF and clarified by centrifugation at 15,000 g for 15 min. The clarified lysates were preabsorbed on protein G-Sepharose (Pharmacia) and then incubated with anti-FLAG-M5 monoclonal antibody (Eastman Kodak Co.) for 2 hours, followed by protein G-Sepharose agarose IgG beads. Immune complexes were washed extensively in the lysis buffer and eluted by boiling in an SDS sample buffer. The eluted proteins were resolved by SDS-PAGE and detected by Western analysis with a horseradish peroxidase-conjugate T7-antibody (Novagen). GST Pull-down assays were performed as described (Ahmad et al., Cancer Res 57:615-9, 1997).

### Example 7

### Subcellular Localization

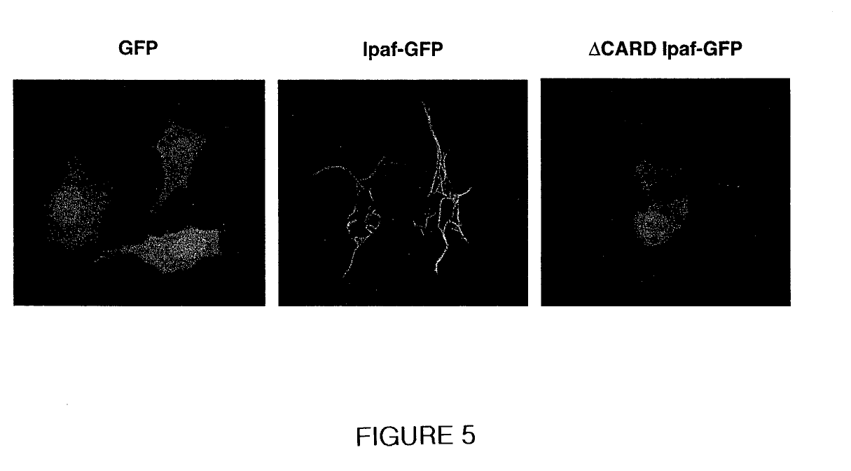

MCF7 cells were transfected with a GFP-tagged Ipaf full length or a CARD-truncated mutant (residues 80-1024). 24 hours following transfection, cells were examined by confocal fluorescent microscopy and photographed. See Example 5. Interestingly, the fusion protein exhibited a cytoplasmic filament pattern (FIG. 5) similar to the death-effector-filaments (DEF) formed by RAIDD, FADD or the death effector domain of procaspase-8 (Siegel et al., J Cell Biol 141, 1243-53, 1998). A similar construct in which the CARD domain of Ipaf has been removed showed mostly diffuse cytoplasmic subcellular localization (FIG. 5). The formation of DEF-like structures by the full length Ipaf but not the CARD-truncated mutant indicates that Ipaf may dimerize or oligomerize in a CARD-mediated manner.

### Example 8

### Self-Interaction of Ipaf Polypeptides

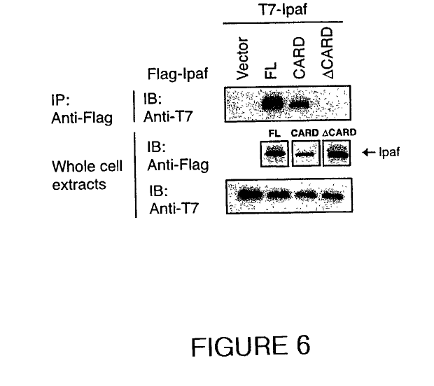

To test if Ipaf polypeptides are able to interact with each other, 293T cells were cotransfected with T7-Ipaf together with one of the following constructs: empty vector (Vector), Flag-tagged full length Ipaf (FL), Flag-tagged CARD domain (residues 1-88) of Ipaf (CARD), or Flag-tagged CARD-deleted mutant (residues 80-1024) of Ipaf (ΔCARD). 24 hours following transfection, cells were lysed and the lysates immunoprecipitated (IP) with an anti-Flag antibody. The immunoprecipitates were immunoblotted (IB) with an anti-T7 antibody. Expression of T7-Ipaf or Flag-tagged constructs was determined by immunoblotting (IB) with anti-T7 or anti-Flag antibodies, respectively. As shown in FIG. 6, the Flag-tagged Ipaf was able to bind to the T7-tagged Ipaf indicating that the Ipaf molecules can indeed associate with each other. This association was dependent on the N-terminal CARD as deletion of this domain prevented the CARD-truncated Ipaf mutant from interacting with the full-length Ipaf. Moreover, the epitope-tagged CARD of Ipaf was able to co-immunoprecipitate the full-length protein.

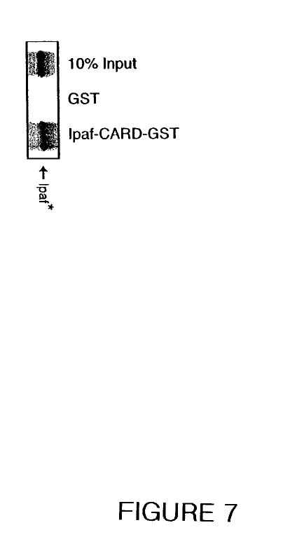

This CARD-mediated interaction was also demonstrated in-vitro as ³⁵S labeled Ipaf was able to associate with an Ipaf-CARD-GST fusion protein but not the GST control (FIG. 7). ³⁵S-labeled Ipaf (lane 1, 10% input) was incubated with an equal amount of glutathione Sepharose beads bound to an equal amount of GST (lane 2) or Ipaf-CARD-GST fusion protein (lane 3). Bound proteins were then eluted and analyzed by SDS-PAGE and autoradiography. Taken together, the above results suggest that the CARD domain of Ipaf mediates its oligomerization.

### Example 9

### Interaction of Ipaf With Procaspase-1

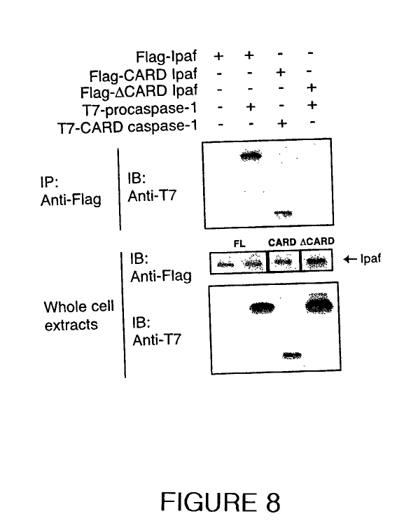

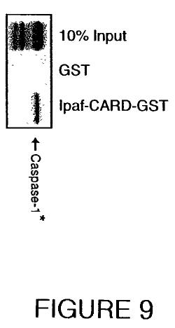

The CARD and its structurally related death effector domain (DED) have been shown to function in diverse signaling pathways that mediate apoptosis (Nunez, et al., (1998) Oncogene 17:3237-3245). Because the CARD of Ipaf shows high similarity to the CARD of procaspase-1, the ability of Ipaf to associate with procaspase-1 was tested in 293T cells. 293T cells were cotransfected with either empty vector or Flag-tagged full length (Flag-Ipaf or FL), CARD domain of residues 1-88 (Flag-CARD Ipaf or CARD) or a CARD domain-deleted mutant of residues 80-1024 (Flag-ΔCARD Ipag or ΔCARD) of Ipaf together with either “empty” vector or T7-tagged full length (T7-procaspase-1) or CARD domain of residues 1-94 (T7-CARD caspase-1) of procaspase-1 expression constructs. 24 hours following transfection, cells were lysed and the lysates immunoprecipitated with an anti-Flag antibody. The immunoprecipitates were immunoblotted with an anti-T7 antibody. The expression of different T7- or Flag-tagged chimeras was determined by immunoblotting with anti-Flag or anti-T7 antibodies, respectively. Immunoprecipitation of epitope-tagged Ipaf transiently expressed in 293T cells quantitatively coprecipited procaspase-1 (FIG. 8). The interaction was mediated by the CARD motifs in the two molecules as truncated Ipaf or procaspase-1 containing only the CARD motifs could interact with each other whereas a truncated version of Ipaf lacking its CARD (ΔCARD) could not precipitate procaspase-1 (FIG. 8). To rule out the possibility that other proteins were necessary for the Ipaf-procaspase-1 interaction, the ability of the Ipaf-CARD-GST fusion protein to associate with in-vitro ³⁵S labeled procaspase-1 was tested. In FIG. 9, ³⁵S-labeled procaspase-1 (lane 1, 10% input) was incubated with an equal amount of glutathione Sepharose beads bound to an equal amount of GST (lane 2) or Ipaf-CARD-GST (lane 3). Bound proteins were then eluted and analyzed by SDS-PAGE and autoradiography. In agreement with a direct interaction, ³⁵S labeled procaspase-1 bound to the Ipaf-CARD-GST fusion protein but not to the GST control (FIG. 9). Of note, no significant interaction was detected between the Ipaf and all other procaspases as well as several other CARD-containing proteins. Combined results suggest that the Ipaf selectively associates with procaspase-1 through a homophilic CARD-CARD interaction.

### Example 10

### Immunodetection of Mature and Precursor Forms of Caspase-1

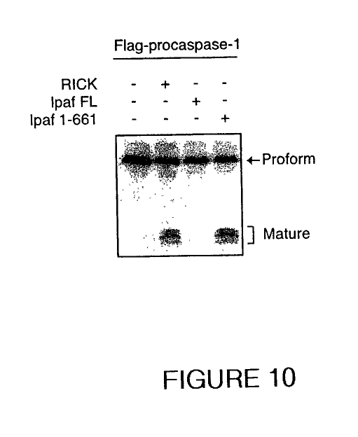

To determine if Ipaf induces procaspase-1 processing, 293T cells were transfected with expression constructs encoding N-terminally Flag-tagged procaspase-1 with or without constructs encoding T7-tagged full length Ipaf or a LRR-truncated variant (Ipaf 1-661). As a positive control, we included an experiment in which procaspase-1 was cotransfected with RICK, which is known to promote procaspase-1 processing (Thome et al., Curr. Biol. 8:885-888, 1998; Humke et al., Cell 103:99-111, 2000). Total lysates from 293T cells (from 100-mm plates) transfected with 1 μg of N-terminally Flag-tagged procaspase-1 in the presence of either 5 μg of RICK or different Ipaf constructs were prepared 18 hours post-transfection and subjected to 15% SDS-PAGE. Tagged caspase-1 was detected by an anti-Flag antibody. Co-expression of the LRR-truncated Ipaf mutant, but not the full-length protein, was able to induce proteolytic activation of procaspase-1 (FIG. 10).

### Example 11

### Apoptosis Assays

293T cells (0.5×10⁵ cells/well) in 12-well plates were transfected with 0.1 μg of LacZ reporter plasmid, 0.2 μg of the caspase-1 plasmid and 0.4 μg of various Ipaf expression plasmids using the LipofectAMINE™ method as per the manufacturer's instruction. Cells were stained for β-galactosidase activity 20 hours following transfection. Normal and apoptotic blue cells were counted. The percentage of apoptotic cells in each experiment was expressed as the mean percentage of stained apoptotic cells as a fraction of the total number of blue cells (n=3).

### Example 12

### Induction of Apoptosis by Ipaf

293T cells were transfected with procaspase-1 plasmid plus β-galactosidase plasmid and either empty vector or the indicated Ipaf constructs together with or without an active site mutant (C285A) procaspase-1 expression construct. 20 hours following transfection cells were fixed and the morphology of 5-bromo-4-chloro-3-indolyl β-D-galactopyranoside-stained cells examined by light microscopy. Data (mean +/− S.E.) shown are the percentage of apoptotic cells among the total number of cells counted (n=3). Ipaf-661, but not the full-length Ipaf, was able to dramatically enhance caspase-1-induced apoptosis in 293T cells (FIG. 11). Expression of full-length Ipaf or Ipaf 1-661 had no effect on cell viability when expressed alone in 293T cells (FIG. 11). These results indicate that Ipaf is a very potent activator of procaspase-1 and that the C-terminal LRR domain of Ipaf acts as a negative regulator of Ipaf activity, as removal of this domain results in a constitutive activation of Ipaf.

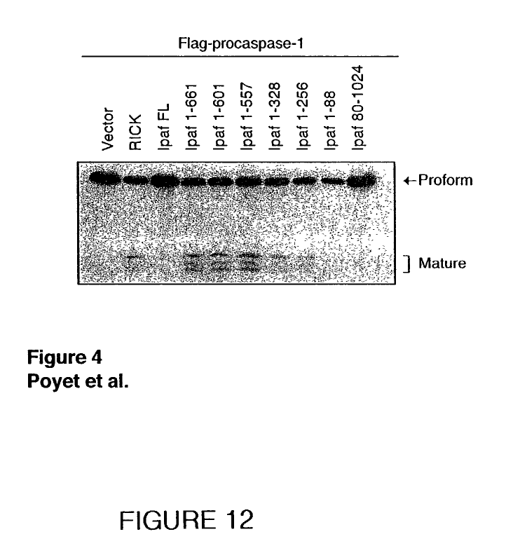

To determine the minimal sequence of Ipaf that is able to promote activation of procaspase-1, engineered mutant forms of Ipaf were tested for their ability to activate procaspase-1. As shown in FIG. 11, co-expression of Ipaf mutants containing the CARD and the NBD (Ipaf 1-557, Ipaf 1-601, Ipaf 1-661) and lacking the LRR with procaspase-1, resulted in massive apoptotic cell death. Additional deletions in the NBD of Ipaf further reduced, but did not abrogate, its activity (FIG. 11). Indeed, coexpression of the CARD motif of Ipaf alone (Ipaf 1-88) with procaspase-1 was sufficient to induce slight increase in apoptotic cell death by caspase-1 (FIG. 11). However, deletion of the CARD motif of Ipaf (Ipaf 80-1024) totally inactivated Ipaf (FIG. 11). In line with these results, only the constitutively active forms, which contain the CARD and entire NBD of Ipaf were able to induce efficient procaspase-1 processing (FIG. 12). The CARD-truncated Ipaf (Ipaf 80-1024) was not able to induce processing of procaspase-1. The separate NBD (Ipaf 80-460) or LRR (Ipaf 661-1024) regions alone were inactive. Combined, the results indicate that the CARD and NBD regions of Ipaf are both necessary and sufficient to bind and activate procaspase-1.

### Example 13

### Cysteine 285 of Procaspase-1 Required for Induction of Processing of Procaspase-1 by Ipaf

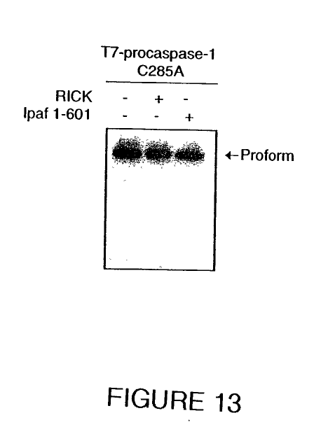

293T cells were cotransfected with an active site mutant (C285A) procaspase-1 expression construct (T7-procaspase-1 C285A) in the presence of either vector control, RICK or Ipaf constructs (i.e., Ipaf 1-601). 18 hours following transfection, processing of the T7-tagged C285A caspase-1 in the total lysates was probed by an anti-T7 antibody. When 293T cells were cotransfected with constructs encoding Ipaf 1-661, wild type procaspase-1 and a procaspase-1 active site mutant (C285A), the procaspase-1 C285A mutant was able to block Ipaf 1-661/caspase-1-induced apoptosis, thus indicating that Ipaf activates specifically procaspase-1 (FIG. 11). As shown in FIG. 13, no processing of the procaspase-1 C285A mutant could be detected in the presence of Ipaf 1-557, nor of RICK. It is therefore likely that Ipaf induces the autoactivation of procaspase-1 by promoting its oligomerization, in accord with the induced proximity model.

### DETAILED DESCRIPTION OF THE INVENTION

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. An isolated nucleic acid molecule comprising a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation or oligomerizes with procaspase-1 or Ipaf of SEQ ID NO: 2.

2. The nucleic acid molecule of claim 1, wherein the Ipaf is a human Ipaf.

3. The nucleic acid molecule of claim 2, wherein the human Ipaf is encoded by the nucleotide sequence of SEQ ID NO: 1 or a degenerate variant thereof.

4. The nucleic acid molecule of claim 1, wherein the encoded Ipaf polypeptide or functional fragment thereof comprises an amino acid sequence of SEQ ID NO: 2.

5. The nucleic acid molecule of claim 1, wherein the polynucleotide sequence encodes a CARD domain of the Ipaf polypeptide, said CARD domain having at least 90% identity with amino acids 1-88 of SEQ ID NO: 2, wherein said CARD domain mediates dimerization between Ipaf polypeptides or with caspase-1/procaspase-1.

6. The nucleic acid molecule of claim 5, wherein the polynucleotide sequence comprises nucleotides 1-264 of SEQ ID NO: 1 or a degenerate variant thereof.

7. The nucleic acid molecule of claim 5, wherein the encoded CARD domain comprises amino acids 1-88 of SEQ ID NO: 2.

8. The nucleic acid molecule of claim 1, wherein the polynucleotide sequence encodes a NBD domain of the Ipaf polypeptide, said NBD domain having at least 90% identity with amino acids 163-457 of SEQ ID NO: 2, wherein said NBD domain is capable of binding nucleotides.

9. The nucleic acid molecule of claim 8, wherein the polynucleotide sequence comprises nucleotides 487-1371 of SEQ ID NO: 1 or a degenerate variant thereof.

10. The nucleic acid molecule of claim 8, wherein the encoded NBD domain comprises amino acids 163-457 of SEQ ID NO: 2.

11. The nucleic acid molecule of claim 1, wherein the polynucleotide sequence encodes an LRR domain of the Ipaf polypeptide, said LRR domain having at least 90% identity with amino acids 656-1024 of SEQ ID NO: 2, wherein said LRR domain is capable of interacting specifically with another LRR domain, thereby mediating protein-protein interactions.

12. The nucleic acid molecule of claim 11, wherein the polynucleotide sequence comprises nucleotides 1966-3072 of SEQ ID NO: 1 or a degenerate variant thereof.

13. The nucleic acid molecule of claim 11, wherein the encoded LRR domain comprises amino acids 656-1024 of SEQ ID NO: 2.

14. The nucleic acid molecule of claim 1, wherein the polynucleotide sequence encodes a functional fragment of the Ipaf polypeptide, wherein the Ipaf functional fragment comprises amino acids 1-256 of SEQ ID NO: 2, and wherein the Ipaf functional fragment induces apoptosis or constitutively induces activation of procaspase-1 or oligomerizes with procaspase-1 or Ipaf of SEQ ID NO: 2.

15. The nucleic acid molecule of claim 14, wherein the polynucleotide sequence comprises nucleotides 1-768 of SEQ ID NO: 1 or a degenerate variant thereof.

16. The nucleic acid molecule of claim 1, wherein the polynucleotide sequence encodes a functional fragment of the Ipaf polypeptide, wherein the Ipaf functional fragment comprises amino acids 1-328 of SEQ ID NO: 2, and wherein the Ipaf functional fragment induces apoptosis or constitutively induces activation of procaspase-1 or oligomerizes with procaspase-1 or Ipaf of SEQ ID NO: 2.

17. The nucleic acid molecule of claim 16, wherein the polynucleotide sequence comprises nucleotides 1-984 of SEQ ID NO: 1 or a degenerate variant thereof.

18. The nucleic acid molecule of claim 1, wherein the polynucleotide sequence encodes a functional fragment of the Ipaf polypeptide, wherein the Ipaf functional fragment comprises amino acids 1-557 of SEQ ID NO: 2, and wherein the Ipaf functional fragment induces apoptosis or constitutively induces activation of procaspase-1 or oligomerizes with procaspase-1 or Ipaf of SEQ ID NO: 2.

19. The nucleic acid molecule of claim 18, wherein the polynucleotide sequence comprises nucleotides 1-1671 of SEQ ID NO: 1 or a degenerate variant thereof.

20. The nucleic acid molecule of claim 1, wherein the polynucleotide sequence encodes a functional fragment of the Ipaf polypeptide, wherein the Ipaf functional fragment comprises amino acids 1-601 of SEQ ID NO: 2, and wherein the Ipaf functional fragment induces apoptosis or constitutively induces activation of procaspase-1 or oligomerizes with procaspase-1 or Ipaf of SEQ ID NO: 2.

21. The nucleic acid molecule of claim 20, wherein the polynucleotide sequence comprises nucleotides 1-1803 of SEQ ID NO: 1 or a degenerate variant thereof.

22. The nucleic acid molecule of claim 1, wherein the polynucleotide sequence encodes a functional fragment of the Ipaf polypeptide, wherein the Ipaf functional fragment comprises amino acids 1-661 of SEQ ID NO: 2, and wherein the Ipaf functional fragment induces apoptosis or constitutively induces activation of procaspase-1 or oligomerizes with procaspase-1 or Ipaf of SEQ ID NO: 2.

23. The nucleic acid molecule of claim 22, wherein the polynucleotide sequence comprises nucleotides 1-1983 of SEQ ID NO: 1 or a degenerate variant thereof.

24. An isolated nucleic acid molecule encoding an Ipaf polypeptide or functional fragment thereof, comprising a polynucleotide sequence having at least 70% identity with SEQ ID NO: 1, wherein said Ipaf polypeptide or functional fragment thereof induces or suppresses procaspase-1 activation or oligomerizes with procaspase-1 or Ipaf of SEQ ID NO: 2.

25. The nucleic acid molecule of claim 24, wherein the polynucleotide sequence comprises a nucleotide sequence of SEQ ID NO: 1 or a degenerate variant thereof.

26. The nucleic acid molecule of claim 24, wherein the polynucleotide sequence comprises at least 20 contiguous nucleotide residues of the sequence provided in SEQ ID NO: 1.

27. The nucleic acid molecule of claim 24, wherein the polynucleotide sequence comprises at least 50 contiguous nucleotide residues of the sequence provided in SEQ ID NO: 1.

28. The nucleic acid molecule of claim 24, wherein the polynucleotide sequence comprises at least 100 contiguous nucleotide residues of the sequence provided in SEQ ID NO: 1.

29. An expression vector comprising:
a promoter, and
an isolated nucleic acid molecule of any one of claims 1 or 24;
wherein the nucleic acid molecule is operably linked to the promoter.

30. The expression vector of claim 29, wherein the promoter is a constitutive or inducible promoter.

31. The nucleic acid molecule of any one of claims 1 or 24, wherein the polynucleotide sequence encoding the Ipaf or functional fragment thereof is fused to a heterologous nucleotide sequence.

32. The nucleic acid molecule of claim 31, wherein the heterologous nucleotide sequence is selected from the group consisting of 1-galactosidase, β-glucuronidase, green fluorescent protein (GFP), blue fluorescent protein (BFP), glutathione-S-transferase (GST), luciferase, horseradish perozidase (HRP), and chloramphenicol acetyltransferase (CAT).

33. The nucleic acid molecule of any one of claims 1 or 24, wherein the polynucleotide sequence encoding the Ipaf or a fragment thereof is fused in frame at either the N-terminal or C-terminal of the Ipaf with a nucleotide sequence encoding a tag, wherein the tag is selected from the group consisting of histidine (His) tags, Asp-Tyr-Lys-Asp-Asp-Asp-Asp-Lys (FLAG) tag (SEQ ID NO: 12), Met-Ala-Ser-Met-Thr-Gly-Gly-Gln-Gln-Met-Gly (T7) tag (SEQ ID NO: 13), influenza hemagglutinin (HA) tags, Myc tags, VSV-G tags, and thioredoxin (Trx) tags.

34. A host cell containing the expression vector of claim 29.

35. The host cell of claim 34, wherein the host cell is selected from the group consisting of a bacterium, a yeast cell, a nematode cell, an insect cell, a plant cell and a mammalian cell.

36. An isolated Ipaf polypeptide or functional fragment thereof having at least 90% amino acid identity with SEQ ID NO: 2, wherein said Ipaf polypeptide or functional fragment thereof induces or suppresses procaspase-1 activation or oligomerizes with procaspase-1 or Ipaf of SEQ ID NO: 2.

37. The Ipaf polypeptide of claim 36, wherein the Ipaf is a human Ipaf.

38. The Ipaf polypeptide of claim 36, wherein the Ipaf polypeptide or functional fragment thereof comprises an amino acid sequence presented in SEQ ID NO: 2.

39. The Ipaf polypeptide of claim 36, wherein the Ipaf polypeptide or functional fragment thereof is encoded by SEQ ID NO: 1, a degenerate variant thereof or fragment thereof.

40. The Ipaf polypeptide of claim 36, wherein the Ipaf polypeptide or functional fragment thereof comprises a CARD domain, said CARD domain having at least 90% identity with amino acids 1-88 of SEQ ID NO: 2; and wherein said CARD domain mediates dimerization between the Ipaf polypeptide or functional fragment thereof derived from SEQ ID NO: 2 or with caspase-1/procaspase-1.

41. The Ipaf polypeptide of claim 40, wherein said CARD domain comprises amino acids 1-88 of SEQ ID NO: 2.

42. The Ipaf polypeptide of claim 40, wherein said CARD domain is encoded by nucleotides 1-264 of SEQ ID NO: 1 or a degenerate variant thereof.

43. The Ipaf polypeptide of claim 36, wherein the Ipaf polypeptide or functional fragment thereof comprises a NBD domain, said NBD domain having at least 90% identity with amino acids 163-457 of SEQ ID NO: 2, wherein said NBD domain is capable of binding nucleotides.

44. The Ipaf polypeptide of claim 43, wherein said NBD domain comprises amino acids 163-457 of SEQ ID NO: 2.

45. The Ipaf polypeptide of claim 43, wherein said NBD domain is encoded by nucleotides 487-1371 of SEQ ID NO: 1 or a degenerate variant thereof.

46. The Ipaf polypeptide of claim 36, wherein the Ipaf polypeptide or functional fragment thereof comprises a LRR domain, said LRR domain having at least 90% identity with amino acids 656-1024 of SEQ ID NO: 2; wherein said LRR domain is capable of interacting specifically with another LRR domain, thereby mediating protein-protein interactions.

47. The Ipaf polypeptide of claim 46, wherein said LRR domain comprises amino acids 656-1024 of SEQ ID NO: 2.

48. The Ipaf polypeptide of claim 46, wherein said LRR domain is encoded by nucleotides 1966-3072 of SEQ ID NO: 1 or a degenerate variant thereof.

49. The Ipaf polypeptide of claim 36, wherein the Ipaf functional fragment comprises amino acids 1-256 of SEQ ID NO: 2, wherein the Ipaf functional fragment induces apoptosis or constitutively induces activation of procaspase-1 or oligomerizes with procaspase-1 or Ipaf of SEQ ID NO: 2.

50. The Ipaf polypeptide of claim 49, wherein the Ipaf functional fragment is encoded by nucleotides 1-768 of SEQ ID NO: 1 or a degenerate variant thereof.

51. The Ipaf polypeptide of claim 36, wherein the Ipaf functional fragment comprises amino acids 1-328 of SEQ ID NO: 2, and wherein the Ipaf functional fragment induces apoptosis or constitutively induces activation of procaspase-1 or oligomerizes with procaspase-1 or Ipaf of SEQ ID NO: 2.

52. The Ipaf polypeptide of claim 51, wherein the Ipaf functional fragment is encoded by nucleotides 1-984 of SEQ ID NO: 1 or a degenerate variant thereof.

53. The Ipaf polypeptide of claim 36, wherein the Ipaf functional fragment comprises amino acids 1-557 of SEQ ID NO: 2, and wherein the Ipaf functional fragment induces apoptosis or constitutively induces activation of procaspase-1 or oligomerizes with procaspase-1 or Ipaf of SEQ ID NO: 2.

54. The Ipaf polypeptide of claim 53, wherein the Ipaf functional fragment is encoded by nucleotides 1-1671 of SEQ ID NO: 1 or a degenerate variant thereof.

55. The Ipaf polypeptide of claim 36, wherein the Ipaf functional fragment comprises amino acids 1-601 of SEQ ID NO: 2, and wherein the Ipaf functional fragment induces apoptosis or constitutively induces activation of procaspase-1 or oligomerizes with procaspase-1 or Ipaf of SEQ ID NO: 2.

56. The Ipaf polypeptide of claim 55, wherein the Ipaf functional fragment is encoded by nucleotides 1-1803 of SEQ ID NO: 1 or a degenerate variant thereof.

57. The Ipaf polypeptide of claim 36, wherein the Ipaf functional fragment comprises amino acids 1-661 of SEQ ID NO: 2, and wherein the Ipaf functional fragment induces apoptosis or constitutively induces activation of procaspase-1 or oligomerizes with procaspase-1 or Ipaf of SEQ ID NO: 2.

58. The Ipaf polypeptide of claim 57, wherein the Ipaf functional fragment is encoded by nucleotides 1-1983 of SEQ ID NO: 1 or a degenerate variant thereof.

59. The Ipaf polypeptide of claim 36, wherein the Ipaf polypeptide comprises at least ten consecutive residues of SEQ ID NO: 2.

60. The Ipaf polypeptide of claim 36, wherein the Ipaf polypeptide comprises at least 20 consecutive residues of SEQ ID NO: 2.

61. An antibody that specifically binds the Ipaf polypeptide or functional fragment thereof of claim 36.

62. The antibody of claim 61, wherein the antibody has an affinity of at least 10⁻⁷M.

63. The antibody of claim 61, wherein the Ipaf polypeptide is a human Ipaf.

64. The antibody of claim 61, wherein the Ipaf polypeptide comprises amino acid sequence of SEQ ID NO: 2.

65. The antibody of claim 61, wherein the antibody specifically binds a CARD domain of the Ipaf polypeptide or functional fragment thereof, said CARD domain having at least 90% identity with amino acids 1-88 of SEQ ID NO: 2; wherein said CARD domain mediates dimerization between the Ipaf polypeptide or functional fragment thereof or procaspase-1.

66. The antibody of claim 65, wherein the CARD domain comprises amino acids 1-88 of SEQ ID NO: 2.

67. The antibody of claim 61, wherein the antibody specifically binds a NBD domain of the Ipaf polypeptide or functional fragment thereof, said NBD domain having at least 90% identity with amino acids 163-457 of SEQ ID NO: 2, wherein said NBD domain is capable of binding nucleotides.

68. The antibody of claim 67, wherein the NBD domain comprises amino acids 163-457 of SEQ ID NO: 2.

69. The antibody of claim 61, wherein the antibody specifically binds a LRR domain of the Ipaf polypeptide or functional fragment thereof, said LRR domain having at least 90% identity with amino acids 656-1024 of SEQ ID NO: 2; wherein said LRR domain is capable of interacting specifically with another LRR domain, thereby mediating protein-protein interactions.

70. The antibody of claim 69, wherein the LRR domain comprises amino acids 656-1024 of SEQ ID NO: 2.

71. The antibody of claim 61, wherein the antibody is a monoclonal, a polyclonal, an antibody fragment, a single chain antibody or a humanized antibody.

72. A cell expressing the antibody of claim 61.

73. A method of identifying an inhibitor or enhancer of an Ipaf mediated caspase processing, comprising:
(a) contacting a sample containing an Ipaf polypeptide or a functional fragment thereof and procaspase-1 or processible fragment thereof with a candidate inhibitor or enhancer, wherein said Ipaf polypeptide or functional fragment thereof has at least 90% amino acid identity with SEQ ID NO: 2, wherein said Ipaf polypeptide or functional fragment thereof induces or suppresses procaspase-1 activation; and
(b) detecting the presence of large and small caspase subunits, and therefrom determining the level of caspase processing activity, wherein a decrease in processing indicates the presence of a caspase processing inhibitor, and wherein an increase in processing indicates the presence of a caspase processing enhancer.

74. The method of claim 73, wherein the procaspase-1 is in vitro translated and labeled.

75. The method of claim 73, wherein the procaspase-1 is labeled, and the label is selected from the group consisting of a radioactive label, a peptide tag, an enzyme and biotin.

76. The method of claim 73, wherein the step of detecting comprises gel electrophoresis.

77. A method of identifying an inhibitor or enhancer of an Ipaf mediated caspase processing, comprising:
(a) contacting a cell transfected with an expression vector of claim 29 with a candidate inhibitor or enhancer; and
(b) detecting the presence of large and small caspase subunits of procaspase-1, and therefrom determining the level of caspase processing activity, wherein a decrease in processing indicates the presence of a caspase inhibitor, and wherein an increase in processing indicates the presence of caspase processing enhancer, thereby identifying an inhibitor or enhancer.

78. The method of claim 77, wherein the step of detecting comprises gel electrophoresis.

79. A method of identifying an inhibitor or enhancer of an Ipaf induced caspase-1 activity, comprising:
(a) contacting a sample containing an Ipaf polypeptide or a functional fragment thereof and procaspase-1 or processible fragment thereof with a candidate inhibitor or enhancer, wherein said Ipaf polypeptide or functional fragment thereof has at least 90% amino acid identity with SEQ ID NO: 2, wherein said Ipaf polypeptide or functional fragment thereof induces or suppresses procaspase-1 activation; and
(b) adding caspase-1 substrate into the sample; and
(c) detecting the turnover of the substrate, wherein the rate of the turnover of the substrate is indicative of the enzymatic activity of the caspase-1, thereby identifying an inhibitor or enhancer.

80. A method of identifying an inhibitor or enhancer of an Ipaf mediated apoptosis, comprising:
(a) contacting a cell transfected with an expression vector of claim 29 with a candidate inhibitor or enhancer; and
(b) detecting cell viability, wherein an increase in cell viability indicates the presence of an inhibitor, and wherein an decrease in cell viability indicates an enhancer, thereby identifying an inhibitor or enhancer.

81. An inhibitor or enhancer identified by any one of the methods of claims 73, 77, 79 and 80.

82. A process for manufacturing an inhibitor or enhancer identified by any one of the methods of claims 73, 77, 79 and 80, comprising:
carrying out the methods of claims 73, 77, 79 and 80 to identify the inhibitor or enhancer; and
derivatizing the inhibitor or enhancer.

83. A process of manufacturing a compound, comprising:
(a) providing an inhibitor or enhancer identified by any one of the methods of claims 73, 77, 79 and 80;
(b) derivatizing the inhibitor or enhancer; and
(c) optionally rescreening the inhibitor or enhancer by any one of the methods of claims 73, 77, 79 and 80.

84. A method of inducing or suppressing apoptosis in a cell, comprising delivering to the cell an effective amount of an isolated nucleic acid molecule encoding an Ipaf polypeptide or functional fragment thereof, under conditions and for a time sufficient for expression of the polypeptide and therefrom detecting apoptosis of the cell; wherein said Ipaf polypeptide or functional fragment has at least 90% identity with SEQ ID NO: 2, and wherein said Ipaf polypeptide or functional fragment thereof induces or suppresses procaspase-1 activation.

85. The method of claim 84, wherein the step of delivering to the cell is selected from the group consisting of injection, transfection, transformation, electroporation, and receptor mediated endocytosis.

86. A method of inducing or suppressing apoptosis in a cell, comprising delivering to the cell an effective amount of an Ipaf polypeptide or functional fragment thereof, under conditions and for a time sufficient to detect therefrom the induction of apoptosis of the cell; wherein said Ipaf polypeptide or functional fragment has at least 90% identity with SEQ ID NO: 2, and wherein said Ipaf polypeptide or functional fragment thereof induces or suppresses procaspase-1 activation.

87. The method of claim 86, wherein the Ipaf polypeptide or functional fragment thereof is a CARD domain of the Ipaf polypeptide, said CARD domain comprising amino acids 1-88 of SEQ ID NO: 2.

88. The method of any one of claims 86-87, wherein the step of delivering to the cell comprises injecting the polypeptide.

89. The method of any one of claims 84-87, wherein the step of apoptosis detection uses a technique selected from the group consisting of altered cellular morphology, DNA fragmentation, annexin binding, caspase cysteine protease activity, and mitochondrial release of cytochrome c.

90. A method of producing an Ipaf polypeptide or functional fragment thereof, comprising culturing a host cell containing an expression vector under conditions and for a time sufficient for expression of the Ipaf polypeptide or functional fragment thereof, wherein the expression vector comprises a nucleic acid molecule encoding the Ipaf polypeptide or functional fragment thereof and a promoter operably linked to the nucleic acid molecule, wherein the nucleic acid molecule comprises a polynucleotide sequence encoding the Ipaf polypeptide or functional fragment thereof, said polypeptide having at least 90% identity with SEQ ID NO: 2, and wherein said Ipaf polypeptide or functional fragment thereof induces or suppresses procaspase-1 activation or oligomerizes with procaspase-1 or Ipaf of SEQ ID NO: 2.

91. The method of claim 90, wherein the nucleic acid molecule consists essentially of SEQ ID NO: 1.

92. An Ipaf polypeptide or functional fragment thereof produced by the method of any one of claims 90 or 91.

93. An antisense nucleic acid molecule comprising a nucleic acid sequence at least 12 nucleotides which is complementary to a nucleic acid molecule encoding an Ipaf polypeptide or functional fragment thereof, wherein the nucleic acid molecule encoding the Ipaf or functional fragment thereof is set forth in SEQ ID NO: 1, and wherein the Ipaf polypeptide or functional fragment thereof induces or suppresses procaspase-1 activation or oligomerizes with procaspase-1 or Ipaf of SEQ ID NO: 2.

94. A ribozyme capable of specifically cleaving a nucleic acid molecule encoding an Ipaf polypeptide or functional fragment thereof, wherein the nucleic acid molecule encoding the Ipaf or functional fragment thereof is set forth in SEQ ID NO: 1, and wherein the Ipaf polypeptide or functional fragment thereof induces or suppresses procaspase-1 activation or oligomerizes with procaspase-1 or Ipaf of SEQ ID NO: 2.

95. A double-stranded RNA molecule comprising a nucleic acid sequence at least 21 nucleotides which is complementary to a nucleic acid molecule encoding an Ipaf polypeptide or functional fragment thereof, wherein the nucleic acid molecule encoding the Ipaf or functional fragment thereof is set forth in SEQ ID NO: 1, and wherein the Ipaf polypeptide or functional fragment thereof induces or suppresses procaspase-1 activation or oligomerizes with procaspase-1 or Ipaf of SEQ ID NO: 2.

96. A gene delivery vehicle comprising a nucleic acid molecule of any one of claims 1 or 24, and a regulatory sequence; wherein the nucleic acid molecule is operably linked to the regulatory sequence.

97. The gene delivery vehicle of claim 96, wherein the vehicle is a virus.

98. The gene delivery vehicle of claim 97, wherein the virus is a retrovirus or adenovirus.

99. The gene delivery vehicle of claim 96, wherein the nucleic acid molecule is associated with a polycation.

100. The gene delivery vehicle of claim 96, further comprising a ligand that binds a cell surface receptor.

101. A method of detecting Ipaf transcription levels, comprising:
(a) providing a polynucleotide probe comprising a sequence that specifically hybridizes to an Ipaf polynucleotide sequence or a complement thereof, wherein the Ipaf polynucleotide sequence is presented in SEQ ID NO: 1.
(b) contacting a biological sample with the probe under hybridization conditions that permit duplex formation;
(c) detecting the presence of the duplex; and
(d) comparing detected levels with a control.

102. The method of claim 101, wherein the polynucleotide probe comprises at least 15 continuous nucleotides of the nucleic acid sequence presented in SEQ ID NO: 1.

103. A method of detecting Ipaf polypeptide levels, comprising:
(a) providing antibodies that specifically binds to an Ipaf polypeptide or fragment thereof, said Ipaf polypeptide or functional fragment thereof having at least 90% amino acid identity with SEQ ID NO: 2;
(b) contacting a biological sample with the antibodies under binding conditions which permit formation of an antibody-polypeptide complex;
(c) detecting the presence of said complex; and
(d) comparing detected levels with a control.

104. A kit for screening for agents that alter apoptosis, comprising an isolated nucleic acid molecule comprising a polynucleotide sequence encoding an Ipaf polypeptide or functional fragment thereof, wherein said Ipaf polypeptide or functional fragment thereof has at least 90% identity with SEQ ID NO: 2, and wherein said Ipaf polypeptide or functional fragment thereof induces or suppresses procaspase-1 activation; and instructions for use of the same.

105. A kit for screening for agents that alter apoptosis, comprising an Ipaf polypeptide or fragment thereof, said Ipaf polypeptide or functional fragment thereof having at least 90% amino acid identity with SEQ ID NO: 2, and a detection reagent that specifically binds to at least one of the foregoing polypeptides.

106. The screening kit of claim 105, wherein the detection reagent is an antibody or antigen-binding fragment thereof.

107. A diagnostic kit comprising:
(a) a polynucleotide probe comprising a portion of the sequence of SEQ ID NO: 1; wherein the polynucleotide probe specifically hybridizes to an Ipaf nucleic acid molecule or a complement thereof when the Ipaf nucleic acid molecule is present in a test biological sample, wherein the Ipaf nucleic acid molecule encodes an Ipaf polypeptide or functional fragment thereof, said Ipaf polypeptide or functional fragment thereof having at least 90% identity with SEQ ID NO: 2; and
(b) instructions for detecting differences between the levels of probe-containing duplexes in the biological sample as compared to a normal biological sample.

108. A diagnostic kit comprising:
(a) an antibody against an Ipaf polypeptide or functional fragment thereof having at least 90% identity amino acid identity with SEQ ID NO: 2, wherein the antibody specifically binds to the Ipaf polypeptide when the polypeptide is present in a test biological sample; and
(b) instructions for detecting differences between the levels of antibody-bound polypeptide in the test biological sample and a normal biological sample.

109. The diagnostic kit of claim 108, wherein the antibody is a monoclonal antibody, a polyclonal antibody, an antibody fragment, a single chain antibody or a humanized antibody.

110. A composition comprising an Ipaf polypeptide or functional fragment thereof, and a physiologically acceptable carrier; wherein said Ipaf polypeptide or functional fragment thereof has at least 90% amino acid identity with SEQ ID NO: 2, and wherein said Ipaf polypeptide or functional fragment thereof induces or suppresses procaspase-1 activation.

111. The composition of claim 110, wherein the Ipaf polypeptide is a human Ipaf consisting essentially of SEQ ID NO: 2 or functional fragment thereof.

112. The composition of claim 111, wherein the Ipaf polypeptide comprises a CARD domain.

113. The composition of claim 111, wherein the Ipaf polypeptide comprises a NBD domain.

114. The composition of claim 111, wherein the Ipaf polypeptide comprises a LRR domain.

115. A composition comprising an antibody that selectively binds to a human Ipaf polypeptide having at least 90% amino acid identity with SEQ ID NO: 2, and a physiologically acceptable carrier, wherein said Ipaf polypeptide induces or suppresses procaspase-1 activation.

116. A composition comprising an Ipaf polynucleotide or fragment or complement thereof, and a physiologically acceptable carrier; wherein said Ipaf polynucleotide encodes an Ipaf polypeptide or functional fragment thereof, said Ipaf polypeptide or functional fragment thereof having at least 90% identity with SEQ ID NO: 2; and wherein said Ipaf polypeptide or functional fragment thereof induces or suppresses procaspase-1 activation.

117. A method of treating a mammal with a disease or disorder associated with an Ipaf polypeptide, comprising administering to the mammal a composition comprising a therapeutically effective amount of a polynucleotide comprising a sequence capable of binding an Ipaf polynucleotide having at least 70% identity with SEQ ID NO: 1, or a complement thereof.

118. The method of claim 117, wherein the polynucleotide is an antisense construct.

119. The method of claim 117, wherein the polynucleotide is a ribozyme construct.

120. The method of claim 117, wherein the polynucleotide is a dsRNA construct.

121. The method of claim 117, wherein the polynucleotide comprises a nucleic acid sequence presented in SEQ ID NO: 1, or a complement thereof.


SEQUENCE LISTING (14 sequences)

SEQ ID NO: 1 - DNA, 3075 bp, Homo sapiens
  CDS (1)...(3075)
atgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaa

SEQ ID NO: 2 - protein, 1024 aa, Homo sapiens
MNFIKDNSRALIQRMGMTVIKQITDDLFVWNVLNREEVNIICCEKVEQDAARGIIHMILKKGSESCNLFLKSLKEWNYPLFQDLNGQSLFHQTSEGDLDDLAQDLKDLYHTPSFLNFYPLGEDIDIIFNLKSTFTEPVLWRKDQHHHRVEQLTLNGLLQALQSPCIIEGESGKGKSTLLQRIAMLWGSGKCKALTKFKFVFFLRLSRAQGGLFETLCDQLLDIPGTIRKQTFMAMLLKLRQRVLFLLDGYNEFKPQNCPEIEALIKENHRFKNMVIVTTTTECLRHIRQFGALTAEVGDMTEDSAQALIREVLIKELAEGLLLQIQKSRCLRNLMKTPLFVVITCAIQMGESEFHSHTQTTLFHTFYDLLIQKNKHKHKGVAASDFIRSLDHCGDLALEGVFSHKFDFELQDVSSVNEDVLLTTGLLCKYTAQRFKPKYKFFHKSFQEYTAGRRLSSLLTSHEPEEVTKGNGYLQKMVSISDITSTYSSLLRYTCGSSVEATRAVMKHLAAVYQHGCLLGLSIAKRPLWRQESLQSVKNTTEQEILKAININSFVECGIHLYQESTSKSALSQEFEAFFQGKSLYINSGNIPDYLFDFFEHLPNCASALDFIKLDFYGGAMASWEKAAEDTGGIHMEEAPETYIPSRAVSLFFNWKQEFRTLEVTLRDFSKLNKQDIRYLGKIFSSATSLRLQIKRCAGVAGSLSLVLSTCKNIYSLMVEASPLTIEDERHITSVTNLKTLSIHDLQNQRLPGGLTDSLGNLKNLTKLIMDNIKMNEEDAIKLAEGLKNLKKMCLFHLTHLSDIGEGMDYIVKSLSSEPCDLEEIQLVSCCLSANAVKILAQNLHNLVKLSILDLSENYLEKDGNEALHELIDRMNVLEQLTALMLPWGCDVQGSLSSLLKHLEEVPQLVKLGLKNWRLTDTEIRILGAFFGKNPLKNFQQLNLAGNRVSSDGWLAFMGVFENLKQLVFFDFSTKEFLPDPALVRKLSQVLSKLTFLQEARLVGWQFDDDDLSVITGAFKLVTA

SEQ ID NO: 3 - protein, 88 aa, Homo sapiens
MNFIKDNSRALIQRMGMTVIKQITDDLFVWNVLNREEVNIICCEKVEQDAARGIIHMILKKGSESCNLFLKSLKEWNYPLFQDLNGQS

SEQ ID NO: 4 - protein, 90 aa, Homo sapiens
MDAKARNCLLQHREALEKDIKTSYIMDHMISDGFLTISEEEKVRNEPTQQQRAAMLIKMILKKDNDSYVSFYNALLHEGYKDLAALLHDG

SEQ ID NO: 5 - protein, 108 aa, Homo sapiens
MEEQGHSEMEIIPSESHPHIQLLKSNRELLVTHIRNTQCLVDNLLKNDYFSAEDAEIVCACPTQPDKVRKILDLVQSKGEEVSEFFLYLLQQLADAYVDLRPWLLEIG

SEQ ID NO: 6 - protein, 90 aa, Homo sapiens
MLCEIECRALSTAHTRLIHDFEPRDALTYLEGKNIFTEDHSELISKMSTRLERIANFLRIYRRQASELGPLIDFFNYNNQSHLADFLEDY

SEQ ID NO: 7 - protein, 91 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSA

SEQ ID NO: 8 - protein, 91 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPN

SEQ ID NO: 9 - protein, 89 aa, Homo sapiens
KKDNHKKKTVKMLEYLGKDVLHGVFNYLAKHDVLTLKEEEKKKYYDAKIEDKALILVDSLRKNRVAHQMFTQTLLNMDQKITSVKPLLQ

SEQ ID NO: 10 - protein, 91 aa, Homo sapiens
MAEDKHNKNPLKMLESLGKELISGLLDDFVEKNVLKLEEEEKKKIYDAKLQDKARVLVDSIRQKNQEAGQVFVQTFLNIDKNSTSIKAPEE

SEQ ID NO: 11 - protein, 5 aa, Unknown
  Caspase large subunit conserved active site sequence.
QACXG

SEQ ID NO: 12 - protein, 8 aa, Artificial Sequence
  Nucleotide sequence encoding tag (FLAG)
DYKDDDDK

SEQ ID NO: 13 - protein, 11 aa, Artificial Sequence
  Nucleotide sequence encoding tag (T7)
MASMTGGQQMG

SEQ ID NO: 14 - DNA, 3219 bp, Homo sapiens
ctgctggaagtcctctgggattcaagaatgaagagtagttttacagaaaaaagaggacaatattgggatcacctttgacctttccatttggaaataatattttctattgtgttatagaaaggtgggaagctttcatccagaacaatgaatttcataaaggacaatagccgagcccttattcaaagaatgggaatgactgttataaagcaaatcacagatgacctatttgtatggaatgttctgaatcgcgaagaagtaaacatcatttgctgcgagaaggtggagcaggatgctgctagagggatcattcacatgattttgaaaaagggttcagagtcctgtaacctctttcttaaatcccttaaggagtggaactatcctctatttcaggacttgaatggacaaagtctttttcatcagacatcagaaggagacttggacgatttggctcaggatttaaaggacttgtaccataccccatcttttctgaacttttatccccttggtgaagatattgacattatttttaacttgaaaagcaccttcacagaacctgtcctgtggaggaaggaccaacaccatcaccgcgtggagcagctgaccctgaatggcctcctgcaggctcttcagagcccctgcatcattgaaggggaatctggcaaaggcaagtccactctgctgcagcgaattgccatgctctggggctccggaaagtgcaaggctctgaccaagttcaaattcgtcttcttcctccgtctcagcagggcccagggtggactttttgaaaccctctgtgatcaactcctggatatacctggcacaatcaggaagcagacattcatggccatgctgctgaagctgcggcagagggttcttttccttcttgatggctacaatgaattcaagccccagaactgcccagaaatcgaagccctgataaaggaaaaccaccgcttcaagaacatggtcatcgtcaccactaccactgagtgcctgaggcacatacggcagtttggtgccctgactgctgaggtgggggatatgacagaagacagcgcccaggctctcatccgagaagtgctgatcaaggagcttgctgaaggcttgttgctccaaattcagaaatccaggtgcttgaggaatctcatgaagacccctctctttgtggtcatcacttgtgcaatccagatgggtgaaagtgagttccactctcacacacaaacaacgctgttccataccttctatgatctgttgatacagaaaaacaaacacaaacataaaggtgtggctgcaagtgacttcattcggagcctggaccactgtggagacctagctctggagggtgtgttctcccacaagtttgatttcgaactgcaggatgtgtccagcgtgaatgaggatgtcctgctgacaactgggctcctctgtaaatatacagctcaaaggttcaagccaaagtataaattctttcacaagtcattccaggagtacacagcaggacgaagactcagcagtttattgacgtctcatgagccagaggaggtgaccaaggggaatggttacttgcagaaaatggtttccatttcggacattacatccacttatagcagcctgctccggtacacctgtgggtcatctgtggaagccaccagggctgttatgaagcacctcgcagcagtgtatcaacacggctgccttctcggactttccatcgccaagaggcctctctggagacaggaatctttgcaaagtgtgaaaaacaccactgagcaagaaattctgaaagccataaacatcaattcctttgtagagtgtggcatccatttatatcaagagagtacatccaaatcagccctgagccaagaatttgaagctttctttcaaggtaaaagcttatatatcaactcagggaacatccccgattacttatttgacttctttgaacatttgcccaattgtgcaagtgccctggacttcattaaactggacttttatgggggagctatggcttcatgggaaaaggctgcagaagacacaggtggaatccacatggaagaggccccagaaacctacattcccagcagggctgtatctttgttcttcaactggaagcaggaattcaggactctggaggtcacactccgggatttcagcaagttgaataagcaagatatcagatatctggggaaaatattcagctctgccacaagcctcaggctgcaaataaagagatgtgctggtgtggctggaagcctcagtttggtcctcagcacctgtaagaacatttattctctcatggtggaagccagtcccctcaccatagaagatgagaggcacatcacatctgtaacaaacctgaaaaccttgagtattcatgacctacagaatcaacggctgccgggtggtctgactgacagcttgggtaacttgaagaaccttacaaagctcataatggataacataaagatgaatgaagaagatgctataaaactagctgaaggcctgaaaaacctgaagaagatgtgtttatttcatttgacccacttgtctgacattggagagggaatggattacatagtcaagtctctgtcaagtgaaccctgtgaccttgaagaaattcaattagtctcctgctgcttgtctgcaaatgcagtgaaaatcctagctcagaatcttcacaatttggtcaaactgagcattcttgatttatcagaaaattacctggaaaaagatggaaatgaagctcttcatgaactgatcgacaggatgaacgtgctagaacagctcaccgcactgatgctgccctggggctgtgacgtgcaaggcagcctgagcagcctgttgaaacatttggaggaggtcccacaactcgtcaagcttgggttgaaaaactggagactcacagatacagagattagaattttaggtgcattttttggaaagaaccctctgaaaaacttccagcagttgaatttggcgggaaatcgtgtgagcagtgatggatggcttgccttcatgggtgtatttgagaatcttaagcaattagtgttttttgactttagtactaaagaatttctacctgatccagcattagtcagaaaacttagccaagtgttatccaagttaacttttctgcaagaagctaggcttgttgggtggcaatttgatgatgatgatctcagtgttattacaggtgcttttaaactagtaactgcttaa